(19) United States
(12) Patent Application Publication
(10) Pub. No.: US 2017/0020854 A1
(43) Pub. Date: Jan. 26, 2017

(54) PRIDOPIDINE BASE FORMULATIONS AND THEIR USE

(71) Applicants: Danit Licht, Givat Shmuel (IL); Ioana Lovinger, Kfar Saba (IL); Muhammad Safadi, Nazareth (IL)

(72) Inventors: Danit Licht, Givat Shmuel (IL); Ioana Lovinger, Kfar Saba (IL); Muhammad Safadi, Nazareth (IL)

(73) Assignee: Teva Pharmaceuticals International GmbH, Jona (CH)

(21) Appl. No.: 15/217,806

(22) Filed: Jul. 22, 2016

Related U.S. Application Data
(60) Provisional application No. 62/195,659, filed on Jul. 22, 2015.

(51) Int. Cl. A61K 31/451; A61K 9/28; A61K 9/48; A61K 9/00

(52) U.S. Cl. CPC A61K 31/451; A61K 9/0053; A61K 9/28; A61K 9/4891

(57) ABSTRACT

This invention provides modified release solid oral dosage form comprising a therapeutically effective amount of pridopidine base, and at least one pharmaceutically acceptable rate controlling excipient.

This application claims the benefit of U.S. Provisional Application No. 62/195,659, filed Jul. 22, 2015, the entire contents of which is hereby incorporated by reference herein.

Throughout this application, various publications are referred by first author and year of publication. Full citations for these publications are presented in a section entitled References immediately preceding the claims. The disclosures of these publications in their entireties are hereby incorporated by reference into this application in order to more fully describe the state of the art to which the invention relates.

### BACKGROUND OF THE INVENTION

Pridopidine

Pridopidine (Huntexil®) is a unique compound developed for the treatment of patients with motor symptoms associated with Huntington's disease. The chemical name of pridopidine is 4-(3-(Methylsulfonyl)phenyl)-1-propylpiperidine and its Chemical Registry Number is CAS 346688-38-8 (CSID:7971505, 2016). The Chemical Registry number of pridopidine hydrochloride is 882737-42-0 (CSID:25948790 2016). Processes of synthesis of pridopidine and a pharmaceutically acceptable salt thereof are disclosed in U.S. Pat. No. 7,923,459. U.S. Pat. No. 6,903,120 claims Pridopidine for the treatment of Parkinson's disease, dyskinesias, dystonias, Tourette's disease, iatrogenic and non-iatrogenic psychoses and hallucinoses, mood and anxiety disorders, sleep disorder, autism spectrum disorder, ADHD, Huntington's disease, age-related cognitive impairment, and disorders related to alcohol abuse and narcotic substance abuse. US Patent Application Publication Nos. 20140378508 and 20150202302, describe methods of treatment with high doses of pridopidine and modified release formulations of pridopidine, respectively.

### BRIEF SUMMARY OF THE INVENTION

This invention provides modified release solid oral dosage form comprising a therapeutically effective amount of pridopidine base, and at least one pharmaceutically acceptable rate controlling excipient.

The invention also provides a modified release solid oral dosage form comprising a therapeutically effective amount of pridopidine base, and at least one pharmaceutically acceptable rate controlling excipient, and wherein the solid oral dosage form provides an in vivo plasma pridopidine concentration profile having a Mean Cₘₐₓ which is lower than a Mean Cₘₐₓ resulting from the b.i.d. administration of an immediate release solid oral dosage form which contains:

a) half the amount of the pridopidine; or
b) between 10% and 49% of the amount of the pridopidine.

The invention further provides a pharmaceutical formulation comprising the modified release solid oral dosage form and one or more pharmaceutically acceptable carriers or excipients. The invention also provides a modified release solid oral dosage form or pharmaceutical formulation which includes an enteric coated tablet or delayed release capsule for use in the treatment of Huntington's Disease, Parkinson's disease, iatrogenic and non-iatrogenic Parkinsonism, dyskinesias, dystonias, Tourette's disease, iatrogenic and non-iatrogenic psychoses and hallucinoses, schizophrenia disorder or schizophreniform disorder, mood and anxiety disorders, manic depressive illness, depression, obsessive-compulsive disease, a sleep disorder, autism spectrum disorder, ADHD, age-related cognitive impairment, abuse of alcohol and substances used as narcotics, Alzheimer's disease or Retts syndrome.

The invention also provides a method of treating a subject afflicted with a condition selected from Huntington's Disease, Parkinson's disease, iatrogenic and non-iatrogenic Parkinsonism, dyskinesias, dystonias, Tourette's disease, iatrogenic and non-iatrogenic psychoses and hallucinoses, schizophrenia disorder or schizophreniform disorder, mood and anxiety disorders, manic depressive illness, depression, obsessive-compulsive disease, a sleep disorder, autism spectrum disorder, ADHD, age-related cognitive impairment, abuse of alcohol and substances used as narcotics, Alzheimer's disease and Retts syndrome, wherein the method comprises administering the modified release solid oral dosage form or pharmaceutical formulation including an enteric coated tablet or delayed release capsule to the subject in need thereof.

The invention also provides a method of treating an individual afflicted with a neurodegenerative disease or a disease related to dopamine, comprising once daily administration of the modified release solid oral dosage form or pharmaceutical formulation including an enteric coated tablet, or delayed release capsule.

The invention also provides for the use of a modified release solid oral dosage form or pharmaceutical formulation including an enteric coated tablet or delayed release capsule for the manufacture of a medicament for treating a subject afflicted with Huntington's Disease, Parkinson's disease, iatrogenic and non-iatrogenic Parkinsonism, dyskinesias, dystonias, Tourette's disease, iatrogenic and non-iatrogenic psychoses and hallucinoses, schizophrenia disorder or schizophreniform disorder, mood and anxiety disorders, manic depressive illness, depression, obsessive-compulsive disease, a sleep disorder, autism spectrum disorder, ADHD, age-related cognitive impairment, abuse of alcohol and substances used as narcotics, Alzheimer's disease or Retts syndrome.

The invention also provides a modified release solid oral dosage form or pharmaceutical formulation including an enteric coated tablet or delayed release capsule wherein the modified release solid oral dosage form or pharmaceutical formulation or enteric coated tablet or delayed release capsule according is adapted for once daily administration.

### BRIEF DESCRIPTION OF THE SEVERAL VIEWS OF THE DRAWINGS

FIG. 1: Pridopidine geometric mean plasma concentrations versus time from Example 1.

FIG. 2: Observed and predicted relation between pridopidine plasma levels and ΔΔQTcF; the line represents population mean predictions. (Circles=45 mg pridopidine; triangles=67.5 mg pridopidine; plus sign=90 mg pridopidine).

FIG. 3: In vitro dissolution rates of the dosage forms MR-1, and MR-2. Each data point for MR-1 is shown by an X and for MR-2 is shown by a dot.

FIG. 4: In vitro dissolution rates of the dosage forms PB-1 compared to MR-1, and MR-2. Each data point for PB-1 is shown by a triangle, for MR-1 is shown by an X and for MR-2 is shown by a dot.

### DETAILED DESCRIPTION OF THE INVENTION

This invention provides modified release solid oral dosage form comprising a therapeutically effective amount of pridopidine base, and at least one pharmaceutically acceptable rate controlling excipient.

In an embodiment, the pridopidine base solid oral dosage form provides an in vivo plasma pridopidine concentration profile having a Mean Cmax of about 1,400 ng/ml or less. In another embodiment, the solid oral dosage form provides an in vivo plasma pridopidine concentration profile having a Mean Cmax of a) about 1,157 ng/ml or less; b) about 906 ng/ml or less; or c) about 499 ng/ml or less. In a further embodiment, the solid oral dosage form provides an in vivo plasma pridopidine concentration profile having a Mean Cmax of: a) about 718 ng/ml or less measured after single dose administration; b) about 486 ng/ml or less measured after single dose administration; or c) about 327 ng/ml or less measured after single dose administration. In another embodiment, the solid oral dosage form provides an in vivo plasma pridopidine concentration profile having a Cmax a) from about 382 ng/ml to about 1,568 ng/ml; b) between 871 ng/ml and 1,568 ng/ml; c) between 382 ng/ml and 1,287 ng/ml; or d) between 639 ng/ml and 1,287 ng/ml. In another embodiment, the solid oral dosage form provides an in vivo plasma pridopidine concentration profile having a Cmax a) from about 244 ng/ml to about 1,002 ng/ml; b) between 244 ng/ml and 813 ng/ml; c) between 493 ng/ml and 1,002 ng/ml; or d) between 324 ng/ml and 813 ng/ml.

In an embodiment, the AUCtau is about 5,253 ng h/ml or more. In another embodiment, the AUC0-inf is about 2,249 ng h/ml or more. In another embodiment, the Mean AUCtau is a) about 7,178 ng h/ml or more; b) about 14,185 ng h/ml or more; or c) about 18,065 ng h/ml or more.

In an embodiment, the Mean AUC0-inf is about a) 5,043 ng h/ml or more; b) about 7,897 ng h/ml or more; or c) about 13,594 ng h/ml or more.

In an embodiment, the in vivo plasma profile is measured at steady state.

In an embodiment, the in vivo plasma profile is measured after single dose administration.

In an embodiment, AUCinf is estimated from AUC0-24.

The invention also provides a modified release solid oral dosage form comprising a therapeutically effective amount of pridopidine base, and at least one pharmaceutically acceptable rate controlling excipient, and wherein the modified release solid oral dosage form provides an in vivo plasma pridopidine concentration profile having a Mean Cmax which is lower than a Mean Cmax resulting from b.i.d. administration of an immediate release solid oral dosage form which contains:

a) half the amount of the pridopidine; or
b) between 10% and 49% of the amount of the pridopidine.

In an embodiment, a) the amount of pridopidine base in the modified release dosage form is more than 45 mg of pridopidine; b) the amount of pridopidine base in the modified release dosage form is at least about 90 mg of pridopidine and the immediate release dosage form contains about 45 mg of pridopidine; c) the amount of pridopidine base in the modified release dosage form is at least about 100 mg of pridopidine and the immediate release solid oral dosage form contains about 45 mg of pridopidine; d) the amount of pridopidine base in the modified release dosage form is at least about 125 mg of pridopidine and the immediate release solid oral dosage form contains about 45 mg of pridopidine; e) the amount of pridopidine base in the modified release dosage form is at least about 135 mg of pridopidine and the immediate release solid oral dosage form contains about 45 mg of pridopidine; f) the amount of pridopidine base in the modified release dosage form is at least about 135 mg of pridopidine and the immediate release solid oral dosage form contains about 67.5 mg of pridopidine; g) the amount of pridopidine base in the modified release dosage form is at least about 150 mg of pridopidine and the immediate release solid oral dosage form contains about 45 mg of pridopidine; h) the amount of pridopidine base in the modified release dosage form is at least about 150 mg of pridopidine and the immediate release solid oral dosage form contains about 67.5 mg of pridopidine; i) the amount of pridopidine base in the modified release dosage form is at least about 180 mg of pridopidine and the immediate release solid oral dosage form contains about 45 mg of pridopidine; j) the amount of pridopidine base in the modified release dosage form is at least about 180 mg of pridopidine and the immediate release solid oral dosage form contains about 67.5 mg of pridopidine; k) the amount of pridopidine base in the modified release dosage form is at least about 180 mg of pridopidine and the immediate release solid oral dosage form contains about 90 mg of pridopidine; 1) the amount of pridopidine base in the modified release dosage form is at least about 200 mg of pridopidine and the immediate release solid oral dosage form contains about 45 mg of pridopidine; m) the amount of pridopidine base in the modified release dosage form is at least about 200 mg of pridopidine and the immediate release solid oral dosage form contains about 67.5 mg of pridopidine; n) the amount of pridopidine base in the modified release dosage form is at least about 200 mg of pridopidine and the immediate release solid oral dosage form contains about 90 mg of pridopidine; o) the amount of pridopidine base in the modified release dosage form is at least about 225 mg of pridopidine and the immediate release solid oral dosage form contains about 45 mg of pridopidine; p) the amount of pridopidine base in the modified release dosage form is at least about 225 mg of pridopidine and the immediate release solid oral dosage form contains about 67.5 mg of pridopidine; q) the amount of pridopidine base in the modified release dosage form is at least about 225 mg of pridopidine and the immediate release solid oral dosage form contains about 90 mg of pridopidine; r) the amount of pridopidine base in the modified release dosage form is at least about 225 mg of pridopidine and the immediate release solid oral dosage form contains about 112.5 mg of pridopidine; s) the amount of pridopidine base in the modified release dosage form is at least about 250 mg of pridopidine and the immediate release solid oral dosage form contains about 45 mg of pridopidine; t) the amount of pridopidine base in the modified release dosage form is at least about 250 mg of pridopidine and the immediate release solid oral dosage form contains about 67.5 mg of pridopidine; u) the amount of pridopidine base in the modified release dosage form is at least about 250 mg of pridopidine and the immediate release solid oral dosage form contains about 90 mg of pridopidine; v) the amount of pridopidine base in the modified release dosage form is at least about 250 mg of pridopidine and the immediate release solid oral dosage form contains about 112.5 mg of pridopidine; w) the amount of pridopidine base in the modified release dosage form is at least about 315 mg of pridopidine and the immediate release solid oral dosage form contains about 45 mg of pridopidine; x) the amount of pridopidine base in the modified release dosage form is at least about 315 mg of pridopidine and the immediate release solid oral dosage form contains about 67.5 mg of pridopidine; y) the amount of pridopidine base in the modified release dosage form is at least about 315 mg of pridopidine and the immediate release solid oral dosage form contains about 90 mg of pridopidine; z) the amount of pridopidine base in the modified release dosage form is at least about 315 mg of pridopidine and the immediate release solid oral dosage form contains about 112.5 mg of pridopidine; or aa) the amount of base in the modified release dosage form is at least about 315 mg of pridopidine and the immediate release solid oral dosage form contains about 157.5 mg of pridopidine.

In an embodiment, the amount of pridopidine base in the modified release dosage form is at least about 90 mg of pridopidine and the immediate release dosage form contains about 45 mg of pridopidine. In another embodiment, the amount of pridopidine in the modified release dosage form is at least about 100 mg of pridopidine and the immediate release solid oral dosage form contains about 45 mg of pridopidine. In another embodiment the amount of pridopidine in the modified release dosage form is at least about 125 mg of pridopidine and the immediate release solid oral dosage form contains about 45 mg of pridopidine. In another embodiment the amount of pridopidine base in the modified release dosage form is at least about 135 mg of pridopidine and the immediate release solid oral dosage form contains about 45 mg to about 67.5 mg of pridopidine.

In an embodiment of the pridopidine base modified release solid oral dosage form, a) the modified release solid oral dosage form provides an in vivo plasma pridopidine concentration profile having a Mean AUCtau which is at least about 50% of the Mean AUCtau provided by the b.i.d. administration of an immediate release solid oral dosage form which contains half the amount of the pridopidine; b) the modified release solid oral dosage form provides an in vivo plasma pridopidine concentration profile having a Mean AUCtau which is at least about 60% of the Mean AUCtau provided by the b.i.d. administration of an immediate release solid oral dosage form which contains half the amount of the pridopidine; c) the modified release solid oral dosage form provides an in vivo plasma pridopidine concentration profile having a Mean AUCtau which is at least about 70% of the Mean AUCtau provided by the b.i.d. administration of an immediate release solid oral dosage form which contains half the amount of the pridopidine; d) the modified release solid oral dosage form provides an in vivo plasma pridopidine concentration profile having a Mean AUCtau which is at least about 80% of the Mean AUCtau provided by the b.i.d. administration of an immediate release solid oral dosage form which contains half the amount of the pridopidine; e) the modified release solid oral dosage form provides an in vivo plasma pridopidine concentration profile having a Mean AUCtau which is at least about 90% of the Mean AUCtau provided by the b.i.d. administration of an immediate release solid oral dosage form which contains half the amount of the pridopidine; or f) the modified release solid oral dosage form provides an in vivo plasma pridopidine concentration profile having a Mean AUCtau which is at least about 95% of the Mean AUCtau provided by the b.i.d. administration of an immediate release solid oral dosage form which contains half the amount of the pridopidine.

In an embodiment, the b.i.d. administration of an immediate release solid oral dosage form has a time interval between doses of 5-10 hours, 6-8 hours, 6.5 hours, or 7 hours.

In an embodiment, the modified release solid oral dosage form provides an in vivo plasma pridopidine concentration profile having a Mean Cmax which is reduced by a percentage compared to the Mean Cmax resulting from the b.i.d. administration of an immediate release dosage form which contains half the amount of the pridopidine, wherein the percentage is at least 5%. In an embodiment, the percentage is a) at least 10%; b) at least 20%; c) at least 30%; d) at least 40%; e) at least 50%; f) at least 60%; g) at least 70%; h) between 10% and 60%; i) between 20% and 50%; j) about 25%; k) about 35%; or l) about 50%.

In an embodiment, the mean time required to reach the maximal plasma, serum or blood concentration of the pridopidine, following administration of the pridopidine is more than 2 hours or more than 4 hours.

In an embodiment, the in vivo plasma profile is measured at steady state. In another embodiment, the in vivo plasma profile is measured after single dose administration. In an embodiment, a) the modified release solid oral dosage form provides an in vivo plasma pridopidine concentration profile having a mean AUC0-inf which is at least about 50% of the mean AUC0-inf provided by the b.i.d. administration of an immediate release solid oral dosage form which contains half the amount of the pridopidine; b) the modified release solid oral dosage form provides an in vivo plasma pridopidine concentration profile having a mean AUC0-inf which is at least about 55% of the mean AUC0-inf provided by the b.i.d. administration of an immediate release solid oral dosage form which contains half the amount of the pridopidine; or c) the modified release solid oral dosage form provides an in vivo plasma pridopidine concentration profile having a mean AUC0-inf which is at least about 75% of the mean AUC0-inf provided by the b.i.d. administration of an immediate release solid oral dosage form which contains half the amount of the pridopidine.

In an embodiment, the modified release solid oral dosage form releases 1-20%, 1-15%, 1-10%, 5%-15%, or 5%-10% of pridopidine after 1 hour when the oral dosage form is placed in an apparatus comprising phosphate buffer having a pH of 6.8.

In an embodiment, the modified release solid oral dosage form releases 1-50%, 5-45%, 10-40%, 10-35%, 10-25%, 10-20% or 15-20% of pridopidine after 3 hours when the oral dosage form is placed in an apparatus comprising phosphate buffer having a pH of 6.8.

In an embodiment, the modified release solid oral dosage form releases 1-70%, 10-60%, 20-50%, 20-45%, 20-40%, 20-35%, or about 35% of pridopidine after 6 hours when the oral dosage form is placed in an apparatus comprising phosphate buffer having a pH of 6.8.

In an embodiment, the modified release solid oral dosage form releases 1-70%, 10-60%, 20-50%, 20-45%, 20-40%, 20-35%, 20-26% or about 25% of pridopidine after 6 hours when the oral dosage form is placed in an apparatus comprising phosphate buffer having a pH of 6.8.

In an embodiment, the modified release solid oral dosage form releases 1-85%, 15-60%, 30-75%, 40-55%, 40-50%, 30-50% or about 48.3% of pridopidine after 9 hours when the oral dosage form is placed in an apparatus comprising phosphate buffer having a pH of 6.8.

In an embodiment, the modified release solid oral dosage form releases 1-95%, 15-90%, 30-80%, 50-70%, 55-65% or about 61% of pridopidine after 12 hours when the solid oral dosage form is placed in an apparatus comprising phosphate buffer having a pH of 6.8.

In an embodiment, the apparatus is a basket and/or paddle apparatus and is maintained at a temperature of 37° C. rotating at 50-100 revolutions per minute.

In an embodiment, the modified release solid oral dosage form releases 0-10%, 0-25%, 0-30%, or 0.5-10%, 7%, or 2.5% of pridopidine after 1 hour when the oral dosage form is placed in an apparatus comprising an acidic medium for one hour. In an embodiment, the modified release solid oral dosage form releases 3-45%, 3-30%, 5-25%, or 5-10% of pridopidine after one hour when the oral dosage form is placed in an apparatus comprising an acidic medium for one hour. In some embodiments, the modified release solid oral dosage form comprises an enteric coated tablet or a delayed release capsule. In some embodiments, the enteric coated tablet comprises the EC PB-1 tablet. In some embodiments, the delayed release capsule comprises the DR PB-1 capsule.

In an embodiment, the modified release solid oral dosage form releases 5%-45% or 5%-30% or 0%-10% or 20%-50% or about 20.5%, or about 7.0% of pridopidine after 2 hours when the oral dosage form is placed in an apparatus comprising an acidic medium for two hours. In an embodiment, the solid oral dosage form releases 1-75%, 5-60%, 10-55%. 25-55%, 10-30%, or 25-35% of pridopidine after 3 hours when the oral dosage form is placed in an apparatus comprising an acidic medium for two hours and then in a phosphate buffer having a pH of 6.8

In an embodiment, the modified release solid oral dosage form releases 5-60%, 10-55%. 25-55%, 10-35%, or 25-35%% of pridopidine after 2 hours when the oral dosage form is placed in an apparatus comprising an acidic medium for two hours and then in a phosphate buffer having a pH of 6.8 for 1 hour.

In another embodiment, the modified release solid oral dosage form releases 1-75%, 3-75%, or 40-60% of pridopidine after 6 hours when the oral dosage form is placed in an apparatus comprising an acidic medium for two hours and then in a phosphate buffer having a pH of 6.8 for 4 hr. In another embodiment, the modified release solid oral dosage form releases 1-70%, 3-70%, or 40-60% of pridopidine after 6 hours when the oral dosage form is placed in an apparatus comprising an acidic medium for two hours and then in a phosphate buffer having a pH of 6.8

In an embodiment, the modified release solid oral dosage form releases 1-90%, 15-75%, or 50-75% of pridopidine after 8 hours when the oral dosage form is placed in an apparatus comprising an acidic medium for two hours and then in a phosphate buffer having a pH of 6.8 for 6 hr. In an embodiment, the modified release solid oral dosage form releases 1-80%, 15-75%, or 50-75% of pridopidine after 8 hours when the oral dosage form is placed in an apparatus comprising an acidic medium for two hours and then in a phosphate buffer having a pH of 6.8.

In an embodiment, the modified release solid oral dosage form releases 1-90%, or 60-85% of pridopidine after 10 hours when the oral dosage form is placed in an apparatus comprising an acidic medium for two hours and then in a phosphate buffer having a pH of 6.8 for 8 hr.

In an embodiment, the modified release solid oral dosage form releases 1-100%, or 60-100% of pridopidine after 12 hours when the oral dosage form is placed in an apparatus comprising an acidic medium for two hours and then in a phosphate buffer having a pH of 6.8 for 10 hr.

In an embodiment, the modified release solid oral dosage form releases less pridopidine after 6 hours when placed in an apparatus comprising phosphate buffer having a pH of 6.8, than a formulation consisting of pridopidine HCl and the same rate controlling excipients when placed under the same conditions, wherein the amount of pridopidine and the amount of rate controlling excipients are the same in the solid oral dosage form and the formulation.

In an embodiment, the modified release solid oral dosage form releases less pridopidine after 6 hours, after 9 hours, or after 12 hours when placed in an apparatus comprising an acidic medium for two hours and then in a phosphate buffer having a pH of 6.8, than a formulation consisting of pridopidine HCl and the same rate controlling excipients when placed under the same conditions, wherein the amount of pridopidine and the amount of rate controlling excipients are the same in the solid oral dosage form and the formulation.

In an embodiment, the acidic medium is 0.1 N HCl. In some embodiments, the acidic medium is not more than 1000 ml of HCl 0.1N. In another embodiment, the acidic medium is gastrointestinal fluids (GIF).

In an embodiment, the apparatus is a basket apparatus maintained at a temperature of 37° C. rotating at 100 revolutions per minute.

In an embodiment, the apparatus is a basket apparatus and/or paddle and is maintained at a temperature of 37° C.

In an embodiment, the modified release solid dosage form comprises from about 45 mg to about 300 mg, or from about 90 mg to about 250 mg, pridopidine.

In an embodiment, the modified release solid dosage form comprises at least about 90 mg, at least about 100 mg, at least about 125 mg, at least about 135 mg, at least about 150 mg, at least about 180 mg, at least about 200 mg, at least about 225 mg, at least about 250 mg, or at least about 315 mg, pridopidine.

In an embodiment, the dosage form comprises about 90 mg, about 100 mg, about 125 mg, about 135 mg, about 150 mg, about 180 mg, about 200 mg, about 225 mg, about 250 mg, or about 315 mg, pridopidine.

In an embodiment, the modified release solid dosage form comprises at about 90 mg, about 100 mg, about 112.5, about 125 mg, about 135 mg pridopidine, about 100-150 mg, about 135-180 mg or about 180-250 mg pridopidine.

In an embodiment, the solid dosage form is a modified release solid dosage form.

In an embodiment, the modified release solid dosage form is in the form of a capsule.

In an embodiment, the modified release solid dosage form is in the form of a tablet.

In an embodiment, the modified release solid dosage form is in the form of a tablet, a mini tablet or a pellet.

In an embodiment, the modified release solid dosage form is in the form of a coated granulate.

In an embodiment, the modified release solid dosage form is an enteric coated dosage form, for example an enteric coated tablet, an enteric coated mini tablet, or an enteric coated pellet.

In an embodiment, the modified release solid dosage form is in the form of delayed release (DR) capsule filled with, for example, pridopidine base granules, one or more tablets, one or more mini tablets or one or more pellets.

In an embodiment, the rate controlling excipient is a polymeric material. In another embodiment, the polymeric material is selected from a group consisting of: hydrogenated castor oil, polyethylene oxide, ethyl cellulose hydroxypropyl methylcellulose (HPMC), hydroxypropyl cellulose (HPC), polyvinyl alcohol (PVA), vinyl alcohol polymer, polycrylates, polymethacrylates, ethyl acrylate-methyl methacrylate copolymers, glyceryl monostearate, and mixtures thereof. In another embodiment, the polymeric material is hydroxypropyl methylcellulose.

In an embodiment, the rate controlling excipient is a combination of two or more polymeric materials. In another embodiment, the polymeric material is hydroxypropyl methylcellulose or hydrogenated castor oil.

In an embodiment, the total amount of the rate controlling excipients is from about 8% to about 70% of the total weight of the modified release solid dosage form, from about 10% to about 50% of the total weight of the modified release solid dosage form, or from about 20% to about 50% of the total weight of the modified release solid dosage form, from about 30% to about 50% or from about 30% to about 40% of the total weight of the modified release solid dosage form.

In an embodiment, the polymeric material is between 10% and 50%, between 20% and 50%, between 30% and 50%, between 30% and 40%, between 35% and 40%, at least 10%, at least 20%, at least 25%, at least 30%, at least 35%, at least 40%, about 33%, about 36%, about 37%, about 38%, or about 40%, by weight of the modified release solid oral dose form.

In an embodiment, the polymeric material is hydroxypropyl methylcellulose or hydrogenated castor oil, and wherein the hydroxypropyl methylcellulose or hydrogenated castor oil is 33-38% by weight of the modified release solid oral dose form.

In an embodiment, the weight ratio of the pridopidine base to the rate controlling excipient is from about 0.2:1 to about 1:1, about 0.3:1 to about 0.8:1, preferably about 0.5:1 to about 0.7:1.

In an embodiment, the modified release solid oral dosage form further comprises a mucoadhesive. In another embodiment, the mucoadhesive is selected from the group consisting of water soluble or water insoluble hydrophilic polymers, polymers that have swellable networks, hydrogels, and polymers with groups that can cross-link with other polymers or with a mucous membrane, preferably the mucoadhesive is polyethylene oxide.

In an embodiment, the pridopidine comprises from about 15% to about 60%, about 25% to about 50%, about 20% to about 25%, about 20%, or about 25%, by weight of the modified release solid dosage form.

Further provided is a pharmaceutical formulation comprising the modified release solid oral dosage form and one or more pharmaceutically acceptable carriers or excipients.

In an embodiment, the pharmaceutically acceptable carriers or excipients are selected from a group consisting of: binder, filler, plasticizer, glidant and lubricant, diluent, and mixtures thereof.

In an embodiment, the binder is selected from a group consisting of: starch, pregelatinized starch, polyethylene oxide, cellulose polymers, hydroxypropylmethyl cellulose, hydroxypropylcellulose, methylcellulose, hydroxyethyl cellulose, polyvinylpyrrolidone, polyvinyl alcohol and mixtures thereof.

In an embodiment, the filler is selected from a group consisting of: microcrystalline cellulose, sugar spheres, lactose, sorbitol, dextrose, sucrose, mannitol, dibasic or tribasic calcium phosphate, calcium sulfate, starch, retalac and mixtures thereof.

In an embodiment, the filler or diluent is microcrystalline cellulose. In another embodiment, the filler or diluent is lactose. In another embodiment, the filler or diluent is silicified microcrystalline cellulose. In an embodiment, the filler or diluent is a mixture of microcrystalline cellulose and lactose. In an embodiment, the filler is present in an amount of between 5% and about 64% by weight of the modified release solid oral dose form, between 10% and about 50% by weight of the modified release solid oral dose form, between 15% and about 45% by weight of the modified release solid oral dose form, between 20% and 40% by weight of the modified release solid oral dose form, between 29 and 34% by weight of the modified release solid oral dose form, about 34% by weight of the modified release solid oral dose form, about 16% by weight of the modified release solid oral dose form, about 17% by weight of the modified release solid oral dose form or about 18% by weight of the modified release solid oral dose form.

In an embodiment, the filler is a mixture of silicified microcrystalline cellulose and lactose and wherein silicified microcrystalline cellulose is between about 14% and about 16% by weight of the modified release solid oral dose form and lactose is between about 15% and about 18% by weight of the modified release solid oral dose form.

In an embodiment, the lactose is Lactose anhydrous or Lactose SD (spray-dried), DC (direct compression).

In an embodiment, the plasticizer is selected from a group consisting of: polyethylene glycol, triethyl citrate, tributyl citrate, glycerin, dibutyl sebacate, triacetin, diethylphthalat and mixtures thereof.

In an embodiment, the plasticizer is triethyl citrate.

In an embodiment, the glidant is selected from a group consisting of: starch, pregelatinized starch, silicone dioxide, colloidal silicone dioxide, talc and mixtures thereof. In another embodiment, the glidant is colloidal silicone dioxide.

In an embodiment, the glidant is present in an amount of between 0.2% and about 4% by weight of the modified release solid oral dose form, between 0.4% and about 3% by weight of the modified release solid oral dose form, or between 0.43% and about 2% by weight of the modified release solid oral dose form.

In an embodiment, the glidant is present in an amount of between 1.7% and about 4% by weight of the solid oral dose form, between 1.7% and about 3% by weight of the modified release solid oral dose form, between 1.7% and about 2% by weight of the modified release solid oral dose form, between 1.7% and 1.8% by weight of the modified release solid oral dose form, about 1.5% by weight of the solid oral dose form, about 1.7% by weight of the modified release solid oral dose form or about 1.8% by weight of the modified release solid oral dose form.

In an embodiment, the lubricant is selected from a group consisting of: sodium stearyl fumarate, stearic acid, magnesium stearate, calcium stearate, zinc stearate, talc, glyceryl behenate, glyceryl monostearate, and mixtures thereof.

In an embodiment, the lubricant is magnesium stearate.

In an embodiment, the lubricant is between 0.3% and about 4% by weight of the modified release solid oral dose form, between 0.5% and about 3% by weight of the modified release solid oral dose form, or between 1.1% and about 2% by weight of the modified release solid oral dose form.

In an embodiment, the lubricant is between 1.7% and about 4% by weight of the modified release solid oral dose form, between 1.7% and about 3% by weight of the modified release solid oral dose form, between 1.7% and about 2.3% by weight of the modified release solid oral dose form, between 1.8% and about 2.2% by weight of the modified release solid oral dose form, about 1.8% by weight of the modified release solid oral dose form, about 1.9% by weight of the modified release solid oral dose form or about 2% by weight of the modified release solid oral dose form.

In an embodiment, modified release solid oral dose form is a tablet and the tablet further comprises an acid resistant envelope.

In an embodiment, the enteric coated tablet comprises a core comprising the pharmaceutical formulation, and an overcoat layer, wherein the overcoat layer completely surrounds the core.

In an embodiment, the overcoat layer comprises a pH sensitive polymer barrier, a coating suspension, an anionic acrylic polymer or any combination thereof.

In an embodiment, the overcoat layer comprises a pH sensitive polymer barrier.

In an embodiment, the overcoat layer comprises a coating suspension.

In an embodiment, the overcoat layer comprises an anionic acrylic polymer.

In an embodiment, the overcoat layer comprises an anionic polymer with methacrylic acid as a functional group.

In an embodiment, the overcoat layer comprises a methacrylic acid, for example Methyl Methacrylate Copolymer [1:1] or a solid poly(methacylic acid-co-ethyl acrylate) 1:1.

In an embodiment, the overcoat layer dissolves slowly in the stomach, but dissolves quickly in the small intestine.

In another embodiment, the overcoat layer dissolves slowly in a medium with a pH of less than 3, but dissolves quickly in a medium with a pH of more than 6. In an embodiment, an overcoat layer dissolves slowly in a medium when little or no dissolution occurs in the medium during the amount of time the overcoat layer is in the medium. In an embodiment, an overcoat layer dissolves quickly in a medium when the overcoat layer dissolves immediately or soon after being placed in the medium.

In an embodiment, the overcoat layer comprises a lubricant. In an embodiment, the lubricant is talc, stearic acid, magnesium stearate. In an embodiment, the lubricant is magnesium stearate.

In an embodiment, the lubricant is present in an amount of between 0.5% and about 4% by weight of the solid oral dose form, between 1.5% and about 2% by weight of the modified release solid oral dose form or about 1.7% by weight of the modified release solid oral dose form.

In an embodiment, the overcoat layer comprises a plasticizer. In an embodiment, the plasticizer is triethyl citrate.

In an embodiment, the plasticizer is present in an amount of between 0.2% and about 5.0% by weight of the modified release solid oral dose form, between 0.5% and about 1.0% by weight of the modified release solid oral dose form or about 0.7% by weight of the modified release solid oral dose form.

In an embodiment, the modified release solid oral dosage form is a delayed release capsule comprising a core comprising the pharmaceutical formulation, wherein the delayed release capsule completely surrounds the core.

In an embodiment, the enteric coated tablet or the delayed release capsule imparts protection to the core so that said core is afforded protection in the acidic pH environment of the stomach (i.e. pH 3 or less) while capable of releasing drug substance in the environment of small intestine (i.e pH of 6.0 or higher.) In an embodiment, the enteric coated tablet can withstand agitation in a basket at 100 rpm in artificial gastric juice having a pH of 1.2 at a temperature of 37° C. releasing less than 10% pridopidine in two hours.

In an embodiment, the delayed release capsule can withstand agitation in a basket at 100 rpm in artificial gastric juice having a pH of 1.2 at a temperature of 37° C. releasing less than 10% pridopidine in two hours.

The invention also provides a modified release solid oral dosage form or pharmaceutical formulation for use in the treatment of Huntington's Disease, Parkinson's disease, iatrogenic and non-iatrogenic Parkinsonism, dyskinesias, dystonias, Tourette's disease, iatrogenic and non-iatrogenic psychoses and hallucinoses, schizophrenia disorder or schizophreniform disorder, mood and anxiety disorders, manic depressive illness, depression, obsessive-compulsive disease, a sleep disorder, autism spectrum disorder, ADHD, age-related cognitive impairment, abuse of alcohol and substances used as narcotics, Alzheimer's disease or Retts syndrome. In some embodiments, the modified release solid oral dosage form or pharmaceutical formulation comprises an enteric coated tablet or delayed release capsule.

The invention also provides a method of treating a subject afflicted with a condition selected from Huntington's Disease, Parkinson's disease, iatrogenic and non-iatrogenic Parkinsonism, dyskinesias, dystonias, Tourette's disease, iatrogenic and non-iatrogenic psychoses and hallucinoses, schizophrenia disorder or schizophreniform disorder, mood and anxiety disorders, manic depressive illness, depression, obsessive-compulsive disease, a sleep disorder, autism spectrum disorder, ADHD, age-related cognitive impairment, abuse of alcohol and substances used as narcotics, Alzheimer's disease and Retts syndrome, wherein the method comprises administering the modified release solid oral dosage form or pharmaceutical formulation or enteric coated tablet or delayed release capsule to the subject in need thereof.

The invention also provides a method of treating an individual afflicted with a neurodegenerative disease or a disease related to dopamine, comprising once daily administration of the modified release solid oral dosage form or pharmaceutical formulation.

In some embodiments, the modified release solid oral dosage form or pharmaceutical formulation comprises an enteric coated tablet or delayed release capsule.

The invention also provides for the use of a modified release solid oral dosage form or pharmaceutical formulation for the manufacture of a medicament for treating a subject afflicted with Huntington's Disease, Parkinson's disease, iatrogenic and non-iatrogenic Parkinsonism, dyskinesias, dystonias, Tourette's disease, iatrogenic and non-iatrogenic psychoses and hallucinoses, schizophrenia disorder or schizophreniform disorder, mood and anxiety disorders, manic depressive illness, depression, obsessive-compulsive disease, a sleep disorder, autism spectrum disorder, ADHD, age-related cognitive impairment, abuse of alcohol and substances used as narcotics, Alzheimer's disease or Retts syndrome. In some embodiments, the modified release solid oral dosage form or pharmaceutical formulation comprises an enteric coated tablet or delayed release capsule.

The invention also provides a modified release solid oral dosage form or pharmaceutical formulation including an enteric coated tablet or delayed release capsule, wherein the modified release solid oral dosage form or pharmaceutical formulation including an enteric coated tablet or delayed release capsule is adapted for once daily administration.

In an embodiment, the pridopidine base is in a solid form.

In some embodiments dissolution of the modified release solid oral dosage form is tested in a dissolution container in 0.1N HCl.

For the foregoing embodiments, each embodiment disclosed herein is contemplated as being applicable to each of the other disclosed embodiments. In addition, the elements recited in pharmaceutical composition embodiments can be used in the method and use embodiments described herein.

Terms:

As used herein, the term “C” refers to the plasma/serum/blood concentration of an active pharmaceutical ingredient, or drug, following administration of the drug, e.g. pridopidine, or a pharmaceutically acceptable salt thereof, in a biological sample, such as a patient sample (e.g., blood, plasma, serum, and cerebrospinal fluid). The concentration of the drug in the biological sample may be determined by any standard assay method known in the art. The term C includes such concentrations measurements as the Cₘᵢₙ, Cₘₐₓ, and Cₛₛ (average steady state concentration), and allows calculation of PK parameters such as AUC. Typically the term C refers to the plasma, serum or blood concentration.

As used herein, steady state refers to the situation in which the amount of drug eliminated at each dose interval equals the dose for that interval. In an embodiment, steady state administration as used herein is reached after 7 days. In an embodiment, steady state administration as used herein is reached after 9 days. In an embodiment, steady state administration as used herein is reached after 14 days. As used herein, the term “Cₘₐₓ” refers to the maximum plasma, serum or blood concentration of a drug, following administration of the drug, e.g. pridopidine, or a pharmaceutically acceptable salt thereof. Cmax measured at steady state is sometimes referred as to C_{max,ss}. “Mean Cₘₐₓ” “C_{max,ss},” and “mean Cₘₐₓ₀₋ₜ” are the mean of the respective Cₘₐₓ measured in a sample of patients. In an embodiment, the sample of patients includes four patients or more. Preferably, the sample should include ten patients or more.

As used herein, the term “Cₘᵢₙ” refers to the minimum plasma, serum or blood concentration of a drug, following administration of the drug, e.g. pridopidine, or a pharmaceutically acceptable salt thereof. Cₘᵢₙ measured at steady state is sometimes referred as to C_{min,ss}.

As used herein, the term “Tₘₐₓ” refers to the time required to reach the maximal plasma, serum or blood concentration (“Cₘₐₓ”) of the drug, following administration of the drug, e.g. pridopidine, or a pharmaceutically acceptable salt thereof. Tₘₐₓ measured at steady state is sometimes referred as to T_{max,ss}.

As used herein, the term “AUC” refers to the area under the plasma, serum or blood concentration versus time curve.

As used herein, the terms “AUCₜ” and “AUC₀₋ₜ” refer to the area under the plasma, serum or blood concentration versus time curve wherein t is the last measured time point.

As used herein, the terms “AUC_{inf}”, “AUC_{0-inf}” “AUC_{∞}”, “AUC_{0-∞}” and AUC_{infinity} refer to the area under the plasma, serum or blood concentration versus time curve extrapolated to infinity.

As used herein, the terms “AUCₜₐᵤ” and “AUC₀₋ₜₐᵤ” refer to the area under the curve for a plasma, serum or blood concentration versus time curve of a drug over one dosing interval, following the administration of the drug such as pridopidine or a pharmaceutically acceptable salt thereof. The area under the curve is measured for a time tau, where tau is the length of the dosing interval. The term AUC_{tau,ss} measures the exposure over the dosing interval at steady state. As use herein, tau is a 24 hours interval, this includes cases in which the drug is administered b.i.d. “Mean AUC,” “Mean AUCₜ,” “Mean AUC₀₋ₜ,” “Mean AUC_{inf},” “Mean AUCₜₐᵤ” and “Mean AUC₀₋ₜₐᵤ” are the mean of the respective AUC measured in a sample of patients. In an embodiment, the sample of patients includes four patients or more. Preferably, the sample should include ten patients or more.

As used herein, “single dose” administration means that the drug is administered over a 24 hours interval, either as once per day (qd) or twice a day (bid).

Without wishing to be bound to theory, the rate of the drug release is determined by combined controlling mechanism of diffusion and/or erosion through or of the coating layer and the matrix and or the gel layer.

As used herein, the term “immediate release” or “IR” means that the escape or release of a drug in the body, such as pridopidine or a pharmaceutically acceptable salt thereof, from a dosage form (tablet, capsule, pellet, etc.) occurs immediately or soon after administration, usually in minutes to a few hours. For example, 80% of the drug may be dissolved over the first hour. In some embodiments, 80% of the drug may be dissolved over the first 30 minutes. The drug is released in a single action and the time of action of the drug is often limited.

As used herein, the term “modified release” or “MR” means that the escape or release of a drug, such as pridopidine or a pharmaceutically acceptable salt thereof, from the dosage form (tablet, capsule, pellet, etc.) has been modified so that the release rate is slower than that in an unmodified or immediate release dosage form. Drug release takes place at a point in time after administration or for a prolonged period after administration or to a specific target in the body. Drug release may occur over several hours or over several days in order to maintain a therapeutically effective plasma concentration of the drug. Modified release encompasses delayed release (release at a time other than immediately after administration), extended release (release over a prolonged time period), sustained release (rate of drug release is sustained over a period of time), and controlled release (rate of drug release is controlled to get a particular drug concentration profile in the body).

As used herein, a slower dissolution profile is one in which the escape or release of a drug from the dosage form is slower, i.e. it takes more time for the drug to be released in a slower dissolution profile than a faster dissolution profile.

As used herein, the term “rate controlling excipient” refers to an excipient or a combination of excipients present in such amounts sufficient to reduce the rate of drug release from a dosage form, such as pridopidine or a pharmaceutically acceptable salt thereof. A rate controlling excipient or a combination thereof controls the rate of drug release from a dosage form.

As used herein, the term “at least one pharmaceutically acceptable rate controlling excipient” or “one or more pharmaceutically acceptable rate controlling excipients” refers to the presence of one, two, three, four, or more rate controlling excipients in the dosage form.

As used herein, to “treat” or “treating” encompasses, e.g., reducing a symptom, inducing inhibition, regression, or stasis of the disorder and/or disease. As used herein, “inhibition” of disease progression or disease complication in a subject means preventing or reducing the disease progression and/or disease complication in the subject.

As used herein, an “amount” or “dose” of pridopidine as measured in milligrams refers to the milligrams of pridopidine base present in a preparation, regardless of the form of the preparation. A dosage of “90 mg pridopidine” means the amount of pridopidine base in a preparation is 90 mg, regardless of the form of the preparation. Thus, when in the form of a salt, e.g. a pridopidine hydrochloride salt, the weight of the salt form necessary to provide a dose of 90 mg pridopidine would be greater than pridopidine mg due to the presence of the salt ion.

As used herein, a “unit dose”, “unit doses” and “unit dosage form(s)” mean a single drug administration entity/entities.

As used herein, “about” in the context of a numerical value or range means±10% of the numerical value or range recited or claimed.

As used herein, the term “once daily” means administering a dose once every 24 hours. As used herein, the term “qd” or “QD” refers to a once daily administration.

As used herein, reference to a total weight of a dosage form refers to the total weight of the dosage form, such as a tablet or a capsule, including any finishing coat.

As used herein, the term “bioavailability” refers to the rate and extent to which an active pharmaceutical ingredient is absorbed from a dosage form and becomes available at the site of action.

A pharmacokinetic parameter or combinations of such parameters indicate the bioavailability of an active pharmaceutical ingredient, such as, pridopidine following administration of pridopidine or a pharmaceutically acceptable salt thereof. Such pharmacokinetic parameters are known to the person skilled in the art. Examples of such parameters include: Cₘₐₓ, AUC, AUCₜₐᵤ, and Tₘₐₓ.

The dosage forms of the present invention are formulated such that the pridopidine base has an in vitro dissolution profile that is slower than that for an immediate release (IR) formulation.

The dosage forms of the present invention may contain immediate release, sustained or extended release or delayed release components, or combinations thereof.

The pridopidine base in the solid oral dosage forms of the present invention can be provided in a modified release form such as modified, controlled, delayed, or extended release (ER) form, with or without an immediate release (IR) component.

Modified release solid dosage forms can be made by, but not limited to, making pellets of different thicknesses so that the thinnest release the drug first and the thickest last, including a slow dissolving matrix or coating, including a non-dissolving coating around a tablet or capsule with small holes to let the drug out (by diffusion or solvation), controlling release of the drug by diffusion through a coating or matrix or by erosion of the matrix or coating by a process dependent on, for example, a particular condition such as the presence of enzymes or a particular pH. Modified release solid dosage forms have higher amounts of the drug than the amount present in an unmodified or immediate release dosage form.

The modified release solid oral dosage form of the present invention is suitable for administration in a one unit dosage form. Oral dosage forms for the purpose of the present invention include capsules, tablets, pellets, granules, powders coated or uncoated and combinations thereof. Optionally, if the dosage form is a capsule, the pridopidine base is provided in the form of coated or uncoated pellets, granules, powders, mini tablets, tablets or capsules.

As used herein, a “polymeric material” includes any polymer. Any suitable polymeric material may be used in accordance with the teachings presented herein. The polymeric material may be any suitable shape and may take any suitable form.

The modified release solid oral dosage forms of the present invention can further comprise one or more mucoadhesives. Mucoadhesives slow the passage of the dosage form through the body so that the dosage form is inside the body during the interval between administrations so that pridopidine or a pharmaceutically acceptable salt thereof is released in the body.

Mucoadhesives are substances that adhere to a biological tissue for an extended period of time by interfacial forces. The biological tissue is a mucous membrane. Mucoadhesion occur when a mucoadhesive contacts and adheres to a membrane by wetting of the mucoadhesive surface or from the swelling of the mucoadhesive. Further adhesion occurs when the mucoadhesive penetrates into the crevice of the membrane surface or when the chains of the mucoadhesive interacts with those of the mucus on the membrane. Suitable mucoadhesive are polymers that are water soluble or water insoluble hydrophilic polymers, polymers that have swellable networks, hydrogels, and polymers with groups that can cross-link with other polymers or with a mucous membrane.

The modified release solid oral dosage forms of the present invention can comprise at least one mucoadhesive with or without an immediate release component. For example, the dosage forms of the present invention can comprise at least one mucoadhesive with only an extended release component.

Silicified microcrystalline cellulose may be any commercially available form of this excipient, for example Prosolv® SMCC 90.

Hydroxypropyl methylcellulose (HPMC) may be any commercially available form of this hydrophilic carrier, for example Methocel™ K100 Premium CR, Methocel™ DC2, Benecel™ ME 233P.

Lactose spray dried (SD), Lactose anhydrous and Lactose monohydrate may be used interchangeable throughout this invention.

Colloidal silicon dioxide (CSD) is a fumed silica generally prepared by vapour-phase hydrolysis of a silicon compound, such as silicon tetrachloride. The product itself is usually a powder which is commercially available from a number of sources, including Degussa, Inc. (under the trade name Aerosil®); Cabot Corporation (under the trade name Cab-O-Sil); Huber Engineered Materials (Huber GL100 and GL200); Wacker (Wacker HDK®); and E.I. DuPont & Co. Colloidal silicon dioxide is also known as colloidal silica, fumed silica, light anhydrous silicic acid, silicic anhydride, and silicon dioxide fumed, among others. A variety of commercial grades of CSD are produced by varying the manufacturing process.

Ethylcellulose may be added to the formulation in the form of dispersion for example, Surelease®.

Pregelatinized Starch may be any commercially available form of this substance, for example Starch 1500®.

LubriTose™ is Lactose plus between 2% and 10% Glyceryl MonoStearate (GMS), LubriTose™ Yellow contains 10% GMS and LubriTose™ blue contains 2% GMS.

Eudragit® L30D55 is a 30% aqueous dispersion of anionic polymers with methacrylic acid as a functional group and its chemical name is Poly(methacrylic acid-co-ethyl acrylate) 1:1. (Evonik I 2015). EUDRAGIT® L 100-55 is a solid substance which contains an anionic copolymer based on methacrylic acid and ethyl acrylate. (Evonik II 2015)

Any delayed release capsule that is a hard capsule with acid resistance may be used. For example, DRCaps® which are commercially available from Capsugel®.

As used herein, an “acid resistant envelope” refers to an outer shell, capsule, cover, coating or layer which delays, reduces, inhibits or prevents release of the active material in acid conditions. DRCaps® and any kind of enteric coating are examples of acid resistant envelopes.

Tablets and capsules, for example, may contain suitable binders, glidants, lubricants, disintegrating agents, coloring agents, flavoring agents, flow-inducing agents, melting agents, and plasticizers. For instance, for oral administration in the dosage unit form of a tablet or capsule, the active drug component can be combined with an oral, non-toxic, pharmaceutically acceptable, inert carrier such as xylose, gelatin, agar, starch, methyl cellulose, dicalcium phosphate, calcium sulfate, mannitol, sorbitol, microcrystalline cellulose and the like. Suitable binders include starch, gelatin, natural sugars such as corn starch, natural and synthetic gums such as acacia, tragacanth, or sodium alginate, povidone, polyvidone, carboxymethylcellulose, hydroxypropyl cellulose, polyethylene glycol, waxes, and the like. Glidants used in these dosage forms include silicon dioxide and the like. Lubricants used in these dosage forms include sodium oleate, sodium stearate, sodium benzoate, sodium acetate, stearic acid, sodium stearyl fumarate, talc and the like. Disintegrators include, without limitation, starch, methyl cellulose, agar, bentonite, xanthan gum, croscarmellose sodium, sodium starch glycolate and the like, suitable plasticizers include triacetin, triethyl citrate, dibutyl sebacate, polyethylene glycol and the like.

The dosage forms of the present invention may further comprise one or more pharmaceutically acceptable carriers or excipients.

Examples of pharmaceutical acceptable excipients are fillers, binders, glidants, plasticizer and lubricants.

Tablets in accordance with this invention can be prepared by conventional mixing, comminution/milling, and tabletting techniques well known in the pharmaceutical formulations industry. The tablet may be obtained by direct compression by punches and dies fitted to a rotary tabletting press, ejection or compression molding, dry or wet granulation followed by compression, or forming a paste and extruding the paste into a mold or cutting the extrudate into short lengths. Preferably, the process used for preparing tablets is direct compression of the blend.

Compression can be accomplished using conventional equipment. Typically, the blend of active ingredients with or without excipients is passed through a roller apparatus for compaction. However, other means for compacting the API mixture, e.g., compaction into slugs (or “slugging”), may be used.

To achieve the desired modified release rates, the modified release dosage form may be formulated as a polymeric coating or matrix.

USP #1 apparatus (basket), is the apparatus 1 described in the United States Pharmacopeia, 29th Edition, chapter 711. The apparatus may be constructed as follows: The assembly consists of the following: a covered vessel made of glass or other inert, transparent material; a motor; a metallic drive shaft; and a cylindrical basket. The vessel is partially immersed in a suitable water bath of any convenient size or placed in a heating jacket. The water bath or heating jacket permits holding the temperature inside the vessel at 37±0.5 during the test and keeping the bath fluid in constant, smooth motion. No part of the assembly, including the environment in which the assembly is placed, contributes significant motion, agitation, or vibration beyond that due to the smoothly rotating stirring element. Apparatus that permits observation of the specimen and stirring element during the test is preferable. The vessel is cylindrical, with a hemispherical bottom and with one of the following dimensions and capacities: for a nominal capacity of 1 L, the height is 160 mm to 210 mm and its inside diameter is 98 mm to 106 mm; for a nominal capacity of 2 L, the height is 280 mm to 300 mm and its inside diameter is 98 mm to 106 mm; and for a nominal capacity of 4 L, the height is 280 mm to 300 mm and its inside diameter is 145 mm to 155 mm. Its sides are flanged at the top. A fitted cover may be used to retard evaporation. The shaft is positioned so that its axis is not more than 2 mm at any point from the vertical axis of the vessel and rotates smoothly and without significant wobble. A speed-regulating device is used that allows the shaft rotation speed to be selected and maintained at the rate specified in the individual monograph, within ±4%. Shaft and basket components of the stirring element are fabricated of stainless steel type 316 or equivalent.

Unless otherwise specified in the individual monograph, a 40-mesh cloth is used. A basket having a gold coating 0.0001 inch (2.5 μm) thick may be used. The dosage unit is placed in a dry basket at the beginning of each test. The distance between the inside bottom of the vessel and the basket is maintained at 25±2 mm during the test.

Pridopidine

Pridopidine is absorbed relatively rapidly after oral administration with tₘₐₓ between 0.5 to 4 hours (Lindskov 2012). After absorption, pridopidine is eliminated partly by urinary excretion, partly by hepatic metabolism, and primarily by N-depropylation via the CYP2Da6 pathway into one main inactive metabolite, 4-(3-(methylsulfonyl)phenyl)piperidine, with an elimination half-life after repeated doses of 10-14 hours. CYP2D6 polymorphisms can be classified according to one of four levels of activity: poor metabolizers (PMs), intermediate metabolizers (IMs), extensive metabolizers (EMs), and ultrarapid metabolizers (UMs). The EM phenotype is expressed by the majority of the population (around 90%). Approximately 5-10% of the Caucasian European and North American population, and 1% of Chinese, Japanese and Korean populations are PMs. PMs inherit two deficient CYP2D6 alleles and, as a result, metabolize drugs at a notably slower rate. The Ultrarapid metabolizers (UM) phenotype is caused by the duplication, multiduplication, or amplification of active CYP2D6 genes, including primarily the CYP2D6*2 allele, but also involving CYP2D6*1 and others. Individuals with the UM phenotype metabolize drugs at an ultrarapid rate. Lastly, individuals who are heterozygous for a defective CYP2D6 gene often demonstrate an IM phenotype with a wide spectrum of metabolic activity that can range from marginally better than the PM phenotype to activity that is close to that of the EM phenotypec (Bernard 2006).

A Phase 2, Dose-Finding, Randomized, Parallel-Group, Double-Blind, Placebo-Controlled Study, Evaluating the Safety and Efficacy of Pridopidine 45 mg, 67.5 mg, 90 mg, and 112.5 mg Twice-Daily Versus Placebo for Symptomatic Treatment in Patients With Huntington's Disease is currently running (Clinicaltrials.gov Clinical Trial Identifier NCT02006472). Therefore, a dosage form comprising pridopidine at these doses with a good safety profile is desirable. In addition, a dosage form administered less frequently than twice a day would increase compliance and would be preferable for the patients and caregivers.

Enteric Coated (EC) Tablets

An enteric coated tablet is a tablet covered with a substance that delays release of the medication until the tablet has passed through the stomach, with little or no dissolution and into the intestine for dissolution (Farlex 2012). After an enteric coated tablet has been swallowed it is transported by peristaltic contractions of the oesophagus into the stomach. The stomach is a pouch-like structure that serves as a food reservoir during early stages of digestion. The stomach churns and gyrates to mix (food/tablets) with digestive secretions such as the enzyme pepsin and hydrochloric acid (stomach acid). However, the EC tablets are not affected by the stomach acid for up to 2 hours. The tablet is then transported into the upper, small intestine, called the duodenum. The “small” intestine is about 20-23 feet long and consists of 3 segments, the duodenum (22 cm or 10 inches long), the jejunum and the ileum. In the small intestine an EC tablet releases its ingredients in the more alkaline environment. The ingredients are generally absorbed through the intestinal wall into the blood stream.

The present invention is illustrated by the following examples, which are not intended to limit the scope of the invention. It will be appreciated that various modifications are within the spirit and scope of the invention.

### EXAMPLES

### Example 1

### Safety of Pridopidine Administration Following Administration of Immediate Release Dosage Forms

Multiple Ascending Dose (MAD) Study

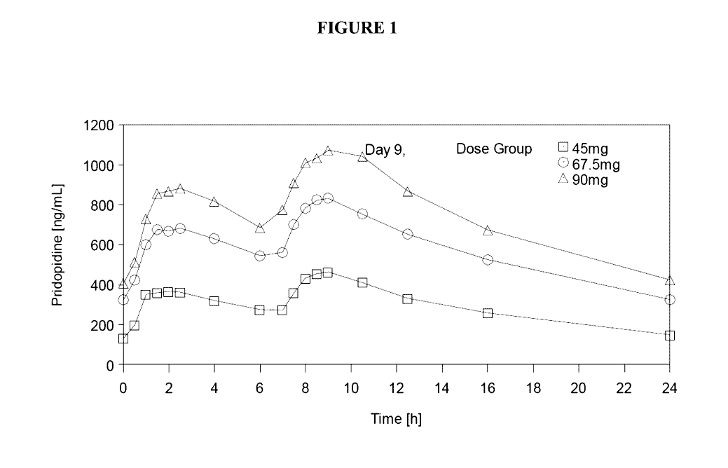

In a Multiple Ascending Dose (MAD) study, thirty-six (36) healthy volunteers of both sexes (age 18-55 years) from the CYP2D6 EM genotype were randomized to 3 cohorts. Within each cohort, 9 subjects were randomized to 2 ascending doses of immediate release (IR) pridopidine HCl b.i.d. in fixed sequence (45-67.5 mg, 67.5-90 mg, and 90-112.5 mg, using 22.5 mg and 45 mg IR tablets), and 3 subjects to matching placebo b.i.d. treatment in both treatment periods. Each period consisted of 9 consecutive days of b.i.d. dosing (with a 6.5 hr interval between the morning and the afternoon dose) to steady state (Osterberg 2012). Pridopidine drug concentrations were monitored up to 24 hours after the first dose and single dose parameters (associated with the first 24 hours interval) were determined. The geometric mean plasma concentrations versus time during the study are presented in FIG. 1.

Safety and tolerability were assessed by monitoring adverse events (AEs), measuring vital signs, electrocardiograms (ECGs), and clinical laboratory values. Pharmacokinetic (PK) parameters of pridopidine were calculated using non-compartmental methods and summarized by descriptive statistics by treatment/dose level (Table 1 and 2 for Day 9 and Day 1, respectively). The dosing interval in this trial (tau) was defined as 24 hours. In Tables 1 and 2, N: Number of subjects; BID:b.i.d.

TABLE 1
Summary of pharmacokinetic parameters
at steady state (mean ± SD)

| | | Mean ± SD | | | |
|---|---|---|---|---|---|
| | | | | | Tmax, |
| | Dose and | AUCtau, ss | Cmax, ss | | ss (h) |
| N | Regimen | (hr*ng/mL) | (ng/mL) | T½ (h) | (range) |
| 8 | IR 45 mg | 7178 ± 1672 | 499 ± 97 | 10.5 ± 3.05 | 1.5 |
| | BID | 5253-10458 | 382-664 | | (1.0-2.5) |
| 16 | IR 67.5 | 14185 ± 3747 | 906 ± 207 | 10.4 ± 2.5 | 2.0 |
| | mg BID | 10228-21065 | 639-1287 | | (1.0-4.0) |
| 14 | IR 90 mg | 18065 ± 3413 | 1157 ± 190 | 10.2 ± 2.1 | 2.0 |
| | BID | 12670-24151 | 871-1568 | | (1.0-4.0) |

TABLE 2
Summary of pharmacokinetic parameters after
single dose administration (mean ± SD)

| | | Mean ± SD | | Median | |
|---|---|---|---|---|---|
| | Dose and | AUC_{0-inf} | Cmax,_{6.5-24} | T½ (h) | Tmax, ss (h) |
| N | Regimen | (hr*ng/mL) | (ng/mL) | (range) | (range) |
| 8 | IR 45 mg BID | 5043 ± 3276 | 327 ± 99.3 | 6.41 | 1.0 |
| | | 2249-12570 | 244-545 | (4.31-15.4) | (1.00-2.50) |
| 16 | IR 67.5 mg BID | 7897 ± 2811 | 486 ± 116 | 7.40 | 1.5 |
| | | 3907-14620 | 324-813 | (4.39-11.2) | (1.00-2.50) |
| 14 | IR 90 mg BID | 13594 ± 3880 | 718 ± 144 | 9.00 | 1.75 |
| | | 7934-22138 | 493-1002 | (6.61-14.0) | (1.00-2.50) |

As shown in Table 3, the adverse events, such as gastrointestinal disorders, increased in frequency with increasing doses. Psychiatric disorders were primarily observed at the 90 mg dose b.i.d., with one observation of psychiatric disorder in the 45 mg dose b.i.d.

A prolonged QT interval has been associated with increased risks for Torsade de Points. Electrocardiogram (ECG) measurements were collected at baseline (predose on the 1^{st} day) and serially on Day 9 (coupled to the PK samples). A high precision QT measurement technique was implemented. The primary endpoint for the QTc analysis was placebo-corrected change-from-baseline QTcF (QT corrected through the Fredericia correction; AAQTcF). The relationship between pridopidine plasma concentrations and AAQTcF was quantified using a linear mixed-effects modeling approach.

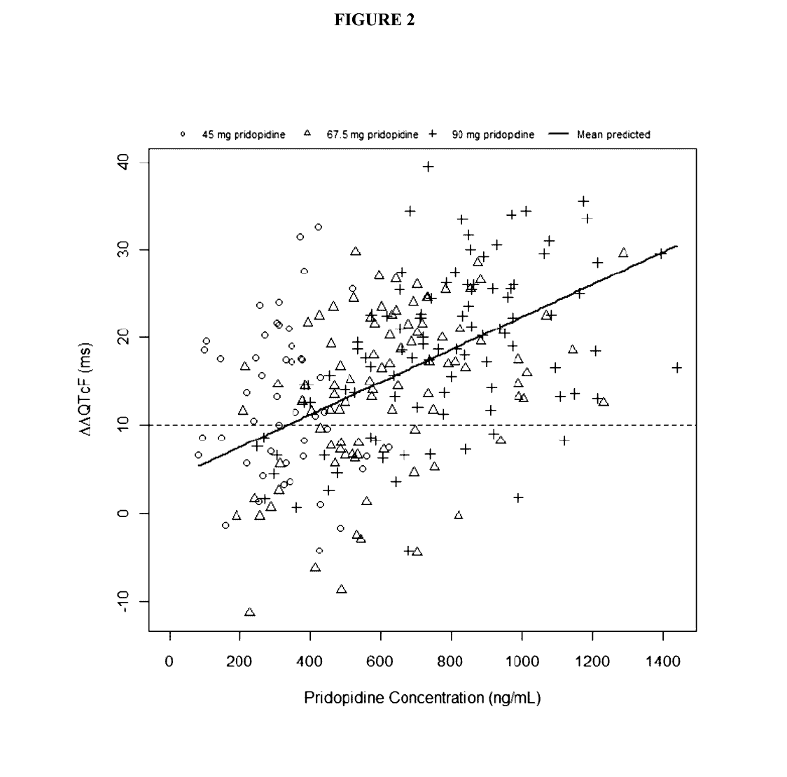

The results showed a concentration-dependent effect of pridopidine on AAQTcF, suggesting that higher concentrations result in longer QT prolongation. The estimated population intercept and slope was 3.82 ms and 0.0185 ms per ng/mL (CI: 0.0139 to 0.0231), respectively (FIG. 2).

TABLE 3
Summary of most common adverse events (>10%)
in selected system organ class of special interest

| | | 45 mg bid | 67.5 mg bid | 90 mg bid |
|---|---|---|---|---|
| | Placebo | pridopidine | priodopidine | pridopidine |
| | N = 14 | N = 9 | N = 17 | N = 18 |
| | N (%) E | N (%) E | N (%) E | N (%) E |
| Nervous system | 8 (57.1%) 17 | 6 (66.7%) 12 | 12 (70.6%) 33 | 14 (77.8%) 39 |
| disorders | | | | |
| Headache | 8 (57.1%) 14 | 4 (44.4%) 8 | 11 (64.7%) 24 | 7 (38.9%) 17 |
| Dizziness | 2 (14.3%) 2 | 1 (11.1%) 1 | 6 (35.3%) 7 | 9 (50.0%) 11 |
| Dysgeusia | 1 (7.1%) 1 | 1 (11.1%) 1 | 1 (5.9%) 1 | 10 (55.6%) 10 |
| Syncope | 0 | 1 (11.1%) 1 | 1 (5.9%) 1 | 0 |
| Paraesthesia | 0 | 1 (11.1%) 1 | 0 | 0 |
| Gastrointestinal | 5 (35.7%) 7 | 1 (11.1%) 2 | 6 (35.3%) 14 | 10 (55.6%) 25 |
| disorders | | | | |
| Nausea | 3 (21.4%) 3 | 0 | 2 (11.8%) 5 | 4 (22.2%) 8 |
| Vomiting | 2 (14.3%) 2 | 0 | 2 (11.8%) 2 | 3 (16.7%) 6 |
| Dry mouth | 1 (7.1%) 1 | 0 | 0 | 5 (27.8%) 5 |
| Diarrhoea | 0 | 0 | 2 (11.8%) 2 | 3 (16.7%) 3 |
| Constipation | 0 | 1 (11.1%) 1 | 0 | 1 (5.6%) 1 |
| Dyspepsia | 0 | 0 | 2 (11.8%) 2 | 0 |
| Faeces hard | 0 | 1 (11.1%) 1 | 0 | 0 |
| Psychiatric disorders | 0 | 1 (11.1%) 1 | 0 | 7 (38.9%) 12 |
| Insomnia | 0 | 0 | 0 | 3 (16.7%) 3 |
| Nightmare | 0 | 0 | 0 | 2 (11.1%) 3 |
| Depressed mood | 0 | 0 | 0 | 2 (11.1%) 2 |
| Emotional disorder | 0 | 1 (11.1%) 1 | 0 | 0 |

N: Number of subjects,
%: percentage of subjects in safety analysis set,
E: Number of events

### Summary of the Results of Example 1

The results as presented in Table 1 showed that a mean C_{max,ss} as high as about 1,157 ng/ml (with a maximal measured value of 1,568 ng/ml), can be safely administered to humans. The results presented in Table 1 also showed that the 45 mg IR b.i.d. administration resulted in a mean C_{max,ss} value of 499 ng/ml and mean AUC_{tau,ss} value (tau defined as a 24 hours interval covering two doses) of 7,178 hr*ng/mL; these values are known to show therapeutic benefit. The range of AUC_{tau,ss} resulting from the administration of 45-90 mg b.i.d was 5,253-24,151 hr*ng/mL. Similarly, the results as presented in Table 2 showed that a mean Cmax as high as about 718 ng/ml at day 1 (with a maximal measured value of 1002 ng/ml), can be safely administered to humans. The results presented in Table 2 also shows that the 45 mg IR bid administration resulted in a mean Cmax value of 327 ng/ml and mean AUC_{0-inf} value of 5043 hr*ng/mL. The range of AUC_{0-inf} resulting from the administration of 45-90 mg b.i.d was 2,249-22,138 hr*ng/mL.

Additionally, the results presented in FIG. 2 show that a concentration as high as 1,400 ng/ml can be considered safe related to the potential prolongation of the QT interval.

The results provided in Tables 1, 2, and 3 show that when certain dosages of pridopidine are administered, there is a risk of increasing the frequency of adverse events in comparison to the frequency of adverse events in previously tested safe dosages of pridopidine. The adverse events include, but are not limited to, QT interval prolongation, gastrointestinal disorders, and psychiatric disorders. The problem to be solved by this application is to provide new formulations of high dose pridopidine base which reduce the frequency of the adverse events. By preventing the Cmax from reaching very high values, applicants can limit the adverse events, such as those shown in Example 1. It was not known that one should prevent the Cmax of pridopidine from peaking in order to minimize some or all adverse events related to a pridopidine dose. Once this problem is understood, applicants developed the present modified release dosage form of pridopidine base which prevents the Cₘₐₓ from rising above previously tested safe doses.

### Example 2

### Modified Release (MR) Pridopidine HCl Dosage Forms

Modified release formulations of MR-1 and MR-2 comprising pridopidine HCl were developed. Modified release formulations MR-1 and MR-2 include 101.6 mg of pridopidine HCl equivalent to 90 mg pridopidine base and involved hydrophilic (hydroxypropyl methylcellulose [HPMC]) or hydrophobic (hydrogenated castor oil [HCO]) carriers. The water-soluble polymer is based on matrix mechanism which involves wetting and hydration and gel layer formation on the outer tablet surface.

TABLE 4
Formulation composition of modified release
solid dose forms using Pridopidine HCl

| | Composition (mg) | | |
|---|---|---|---|
| | Batch No. | MR-1 | MR-2 |
| Formulation Ingredients | Use | mg/Tablet | mg/Tablet |
| Pridopidine HCl | Drug | 101.6 | 101.6 |
| | Substance | | |
| Silicified Microcrystalline | Diluent | 63.2 | 63.2 |
| Cellulose (Prosolv ® SMCC 90) | or filler | | |
| Hydroxypropyl methylcellulose | Hydrophilic | ** | 150.0 |
| (HPMC) (Methocel ™ K100M | carrier | | |
| Premium CR/Methocel DC2) | | | |
| Hydrogenated Castor Oil (HCO) | Hydrophobic | 150.0 | ** |
| | carrier | | |
| Lactose SD, DC | Filler | 70.0 | 70.0 |
| Colloidal Silicon Dioxide | Flow agent | 7.2 | 7.2 |
| (Aerosil ®) | | | |
| Magnesium Stearate | Lubricant | 8.0 | 8.0 |
| Tablet Weight | | 400.0 | 400.0 |
| Dissolution profile | | 9-12 h | 9-12 |
| | | release | release |

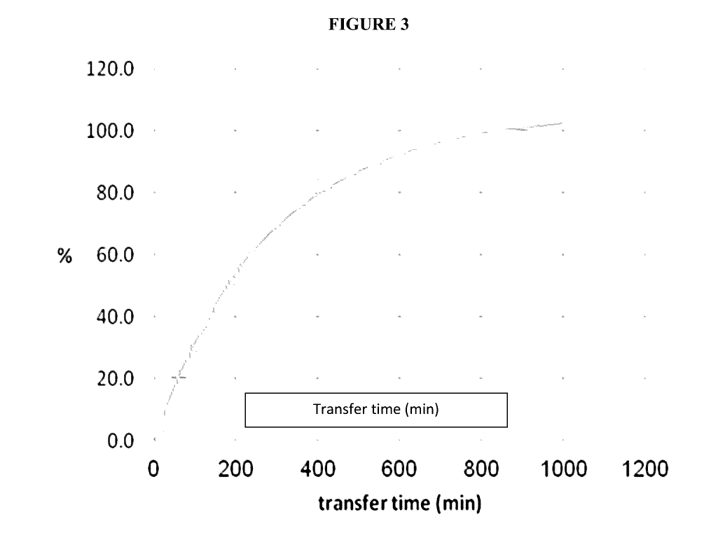

Those formulations provide a dissolution profile within 12 hours. The in vitro dissolution results are presented in Table 5 and FIG. 3.

TABLE 5
Dissolution rate of pridopidine HCl modified release solid
dosage forms MR-1 and MR-2 in phosphate buffer pH 6.8

| | | Time | | | | | |
|---|---|---|---|---|---|---|---|
| | Batch No. | 5 min | 1 h | 3 h | 6 h | 9 h | 12 h |
| Release | MR-1 | 1.3 | 20.5 | 50.3 | 75.7 | 89.3 | 97.0 |
| rate (%) | MR-2 | 4.6 | 27.6 | 54.9 | 78.4 | 87.8 | 97.9 |

TABLE 6
Dissolution rate of pridopidine HCl modified release
solid dosage forms MR-1 and MR-2 in 2 hours in 0.1N
HCl followed by phosphate buffer pH 6.8

% dissolved

| Sampling time | | | | MR-3 (8 mg |
|---|---|---|---|---|
| (minutes) | pH | MR-1 | MR-2 | ethylcellulose tablet) |
| 60 | 1.2 | 41 | 35 | 9 |
| 120 | 1.2 | 57 | 54 | 24 |
| 180 | 6.8 | 68 | 67 | 37 |
| 240 | 6.8 | 75 | 76 | 48 |
| 360 | 6.8 | 86 | 88 | 64 |
| 480 | 6.8 | 92 | 96 | 77 |
| 600 | 6.8 | 97 | 101 | 86 |
| 720 | 6.8 | | | 94 |

### Example 3

### Modified Release Pridopidine Base with the Formulations of Example 2

A modified release solid oral formulation which comprises pridopidine base is described in this example. In example 2, the modified release formulations comprised pridopidine hydrochloride salt. Table 7 provides an example of 90 mg pridopidine base modified release tablet.

TABLE 7
90 mg Pridopidine Base Modified Release Tablet

| | Use | PB-1 |
|---|---|---|
| Formulation Ingredients | — | mg/Tablet |
| Pridopidine Base | Drug | 90.0 |
| | Substance | |
| Silicified Microcrystalline | Diluent or | 63.2 |
| Cellulose (Prosolv ®) | filler | |
| Hydroxypropyl methylcellulose (HPMC) | Hydrophilic | 150.0 |
| (Methocel ™ K100M Premium) | carrier | |
| Lactose SD, DC | Filler | 70.0 |
| Colloidal Silicon Dioxide (Aerosil ®) | Flow agent | 6.8 |
| Mg. Stearate | Lubricant | 8.0 |
| DRcaps ® | Capsules | — |
| Eudragit ® L30D55 | Coating | — |
| | suspension | |
| Talc | Lubricant | — |
| Triethyl Citrate | Plasticizer | |
| Tablet Weight | | 388.0 |

Dissolution Tests of the Dosage Forms:

1) Dissolution in HCl followed by phosphate buffer, pH 6.8

A typical dissolution assay for pridopidine HCl tablets or pridopidine base tablets uses an USP #1 apparatus (basket), rotating at 100 RPM and 37° C. in 500 mL of HCl 0.1N for 2 hours followed by dissolution in buffer phosphate pH 6.8, for 12 hours. The buffer phosphate is prepared by dissolving 6.805 g of KH₂PO₄ phosphate dibasic and 4.48 mL 5M NaOH, diluted to 1000 mL with deionized water and mixed thoroughly. In dissolution tests without an acidic medium, the tablet or capsule is placed directly in a buffer phosphate solution. The sample is tested by UV detector set at 268 nm and then returned to the dissolution vessel. The same dissolution results were obtained using an USP #2 apparatus (paddle) at 75 RPM.

The dissolution rate of the formulation comprising pridopidine base (PB-1) was compared with dissolution rates of modified release formulations comprising pridopidine HCl (MR-1 and MR-2). Results are presented in Table 8.

The results presented in Table 8 show that using pridopidine base as in Formulation PB-1 provided a slower dissolution rate of pridopidine in buffer phosphate pH 6.8 compared to formulations MR-1 and MR-2 in HCl+ buffer phosphate pH 6.8.

However, in acidic medium the dissolution rate was higher and more similar to the dissolution results for MR-1 and MR-2 (Table 8). Without wishing to be bound to theory pridopidine base is transformed into its hydrochloride salt in the assay conditions.

TABLE 8
Dissolution Rate of MR-1 and MR-2 tablets compared
to PB-1 Tablets in phosphate buffer pH 6.8 and
in 0.1N HCl and phosphate buffer pH 6.8.

| | | Time | | | | | |
|---|---|---|---|---|---|---|---|
| | Batch No. | 5 min | 1 h | 3 h | 6 h | 9 h | 12 h |
| in HCl | MR-1 | 1.3 | 20.5 | 50.3 | 75.7 | 89.3 | 97.0 |
| and | Pridopidine | | | | | | |
| Buffer* | HCl | | | | | | |
| | MR-2 | 4.6 | 27.6 | 54.9 | 78.4 | 91.1 | 97.9 |
| | Pridopidine | | | | | | |
| | HCl | | | | | | |
| in buffer | PB-1 | 0 | 8.1 | 19.4 | 24.5 | 48.3 | 61.0 |
| only | Pridopidine | | | | | | |
| | Base | | | | | | |
| in HCl | PB-1 | | 24.9 | 51.7 | 66.7 | 75.7 | 83.8 |
| and | Pridopidine | | | | | | |
| Buffer* | Base | | | | | | |

*2 hours in HCl and more than 10 hours in phosphate buffer pH 6.8.

Therefore, in order to avoid this phenomenon, which mimics the gastric milieu (pH<3), the pridopidine base core tablet (PB-1) was coated with an enteric coating (EC) polymer (EC-PB-1), or encapsulated in a delayed release (DR) capsule (DR-PB-1) which are resistant in the stomach under acidic conditions (Table 9).

The enteric coating contains a pH sensitive polymer barrier, which remains intact in the acidic environment of the stomach (pH 1.5-3.5), protecting the contents of the tablet. The enteric coating then disintegrates in the small intestine (duodenum), which has an alkaline environment (pH 6.5-7.6). The coating of the pridopidine base enteric coating product (EC-PB-1) is based on an anionic polymer with methacrylic acid as a functional group (Eudragit® L). This coating has been proven for both safety and efficacy. A methacrylic acid-methyl methacrylate copolymer [1:1] (Eudragit®) was chosen as one option of appropriate enteric polymer for the enteric coating. A variety of anionic acrylic polymer grades (Eudragit® grades) are available for enteric applications. A solid poly(methacylic acid-co-ethyl acrylate) 1:1 (Eudragit® L-100-55) forms an enteric coating that dissolves quickly in the small intestine and may be used for the dosage forms disclosed herein.

In addition, a plasticizer was used to increase the flexibility of the film coating. Triethyl citrate was chosen since it is water soluble. In general, the use of hydrophilic plasticizers produces coatings with higher permeability and faster dissolution than lipophilic ones which reduce permeability and dissolution rate. Triethyl citrate combined with a solid poly(methacylic acid-co-ethyl acrylate) 1:1 (Eudragit® L-100-55) promoted the reduced release of pridopidine base in acidic conditions (0.1 N HCl) and release in higher pH conditions (phosphate buffer pH 6.8). Lastly, talc extra fine was added as lubricant and glidant.

TABLE 9
Pridopidine base Modified Release Dosage Forms

| | Batch No. | | | |
|---|---|---|---|---|
| | | | | EC-PB-1 |
| | | | DR-PB-1 | EC Tablets |
| | Use | PB-1 | DR Capsules | (cores: PB-1) |
| Composition | — | mg/Tablet | mg/Capsule | mg/Tablet |
| Pridopidine Base | Drug | 90.0 | 90.0 | 90.0 |
| | Substance | | | |
| Silicified Microcrystalline | Diluent | 63.2 | 63.2 | 63.2 |
| Cellulose (Prosolv ®) | | | | |
| Hydroxypropyl Methyl Cellulose | Hydrophilic | 150.0 | 150.0 | 150.0 |
| (HPMC), Methocel K100 PR CR/DC2 | carrier | | | |
| Lactose SD, DC | Filler | 70.0 | 70.0 | 70.0 |
| Colloidal Silicon Dioxide (Aerosil ®) | Flow agent | 6.8 | 6.8 | 6.8 |
| Mg Stearate | Lubricant | 8.0 | 8.0 | 8.0 |
| delayed release capsule (DR caps ®) | Capsules | — | 70.0 | — |
| Eudragit ® L30D55 | Coating | — | — | 14.6 |
| | suspension | | | |
| Talc | Lubricant | — | — | 7.0 |
| Triethyl Citrate | Plasticizer | | | 2.9 |
| Total tablet/capsule weight | | 388.0 | 458.0 | 412.5 |

Dissolution Rate of Tablets/Capsules based on pridopidine base were tested in HCl buffer for 2 hours followed by a basic environment (phosphate buffer pH 6.8), mimicking the path of the drug product though the stomach and the small intestine. The results are presented in Table 10.

TABLE 10
Dissolution Rate of Pridopidine Base Modified Release
Dosage Forms (comparison with and without HCl buffer)

| | | | | PB-1 (in | | DR-PB-1 | EC-PB-1 |
|---|---|---|---|---|---|---|---|
| | | Total | MR-2 | phosphate | | delayed | Enteric |
| Buffer | Time in | Time | (pridopidine | Buffer pH | | release | Coated |
| stage | pH 6.8 | (min) | HCl) | 6.8 only) | PB-1 | capsules | Tablets |
| HCl | | 0 | | N.A. | 0.0 | 0.0 | 0.0 |
| 0.1N | | 10 | | N.A. | 7.7 | 0.3 | 0.0 |
| | | 20 | | N.A. | 12.1 | 0.5 | 0.2 |
| | | 30 | | N.A. | 15.8 | 1.3 | 0.7 |
| | | 60 | 27.6 | N.A. | 24.9 | 7 | 2.5 |
| | | 90 | | N.A. | 32.8 | 14 | 4.6 |
| | | 120 | 42.0 | N.A. | 39.9 | 20.5 | 7.0 |
| Phosphate | 30 | 150 | | 4.8 | 47.0 | 26.2 | 20.2 |
| Buffer | 60 | 180 | 54.9 | 8.1 | 51.7 | 31.5 | 26.5 |
| pH 6.8 | 90 | 210 | | 11.1 | 55.5 | 36.5 | |
| | 120 | 240 | 64.6 | 14.0 | 58.4 | 41.2 | 34.3 |
| | 180 | 300 | | 19.4 | 62.9 | 49.9 | 39.3 |
| | 240 | 360 | 78.4 | 24.5 | 66.7 | 58.0 | 44.0 |
| | 300 | 420 | | 29.6 | 70.0 | 65.3 | 48.9 |
| | 360 | 480 | | 34.5 | 72.9 | 72.1 | 53.3 |
| | 420 | 540 | 91.1 | 39.2 | 75.7 | 78.4 | 57.5 |
| | 480 | 600 | | 43.8 | 78.6 | 83.6 | 61.8 |
| | 540 | 660 | | 48.3 | 81.8 | 88.3 | 66.0 |
| | 600 | 720 | 97.9 | 52.7 | 83.8 | 92.0 | 69.9 |
| | 660 | 780 | | 57.0 | 86.3 | 100.9 | 73.7 |
| | 720 | 840 | | 61.0 | 88.8 | | 77.3 |
| | 900 | 1020 | | | 95.2 | | 87.4 |
| | 1080 | 1200 | | | | | 95.5 |

Comparison of the release rates showed that the pridopidine base formulation PB-1 incubated in phosphate buffer (pH 6.8) had a slower release profile than the pridopidine HCl incubated in an acidic environment for 2 hours (HCl buffer) followed by a phosphate buffer (pH=6.8) (83.8 vs. 97.9% release after 12 h, respectively). As shown in Table 10, the pridopidine base formulation PB-1 showed a slower release rate when incubated in phosphate buffer only compared to incubation in HCl buffer for 2 hours and then in phosphate buffer (pH=6.8) (61.0 vs. 83.8% release after 12 h, respectively). The release rate of the enteric coated pridopidine base was slower than the PB-1 formulation in the same conditions (69.9 vs. 83.8% release after 12 h, respectively). Moreover, 95.5% pridopidine release was obtained only after 20 hours from the enteric coated pridopidine base. The release rate of all pridopidine formulations containing pridopidine base (PB-1; DR-PB-1 (DR Caps®); EC-PB-1 (enteric coated tablets) was slower than the MR-1 formulation comprising pridopidine HCl in the conditions mimicking transfer of the dosage form through the stomach and intestine (83.8, 92.0 and 69.9% vs. 97.9% after 12 h, respectively).

The results show that when the pridopidine base formulation (PB-1) is transferred through an HCl buffer incubation phase, a great part (39.9%) of the pridopidine base is released in the 2 h incubation stage in HCl buffer. However, when pridopidine base formulated in delayed release (DR) caps (such as DRCaps®) or EC (EC-PB-1 enteric coated tablets) were incubated in HCl buffer for 2 h and then in phosphate buffer pH=6.8, only about 20.5% and 7% of the pridopidine base is released in the HCl buffer stage, respectively.

The prevention of initial high release by the DR Caps and EC coating has safety advantages since immediate high release may be related to certain cardiac safety issues as shown in Example 1.

EC and DR formulations are advantageous for several reasons. First, EC and DR formulations offer design flexibility, the amount of the enteric coating or the amount of the capsule shell will control the dissolution release profile of pridopidine base. Second, the amount and nature of plasticizer (hydrophilic or lipophilic) in the EC formulation or the capsule shell will control the dissolution release profile of pridopidine. Third, the amounts and nature of polymer (hydrophilic or lipophilic) in the EC formulation or the capsule shell will control the dissolution release profile of pridopidine.

This flexibility allows for the reduction of the size of capsule size or tablet size when needed by reducing polymer amounts. This is important in high dose administration of a tablet or capsule. It is also important in the administration of the capsule or tablet to patients with movement disorders and problems swallowing. For example, if polymer amounts are reduced, and EC or DR and pridopidine base are used, a smaller tablet size is possible with the same release rate as a large pridopidine HCl tablet with the same amount of pridopidine. Therefore, a higher dose without enlarging the tablet size or a reduced tablet size will be available. 2) Dissolution in phosphate buffer, pH 6.8

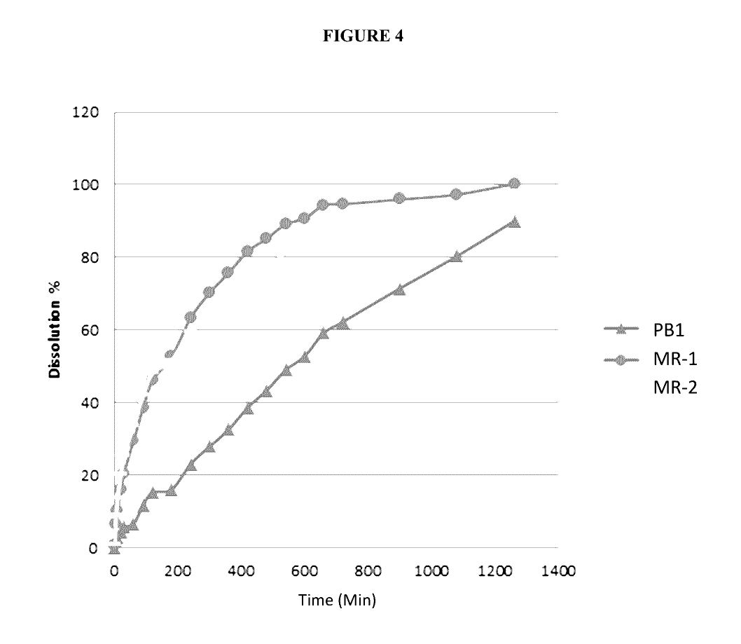

The MR-1, MR-2 and PB-1 tablets were dissolved in a dissolution assay, similar to that described above but in phosphate buffer pH 6.8. Results are shown in Table 11 and in FIG. 4.

TABLE 11
Dissolution profiles of pridopidine base formulation compared
to pridopidine HCl formulations in phosphate buffer pH 6.8

| Time | | | |
|---|---|---|---|
| (Min) | PB-1 | MR-1 | MR-2 |
| 0 | 0.1 | 1 | 0.6 |
| 60 | 6.5 | 33.9 | 29.6 |
| 120 | 15.2 | 48.3 | 46.2 |
| 240 | 23.6 | 59.7 | 63.4 |
| 360 | 32.7 | 68.7 | 75.8 |
| 480 | 43.5 | 77.2 | 85.35 |
| 600 | 52.9 | 83.4 | 90.8 |
| 720 | 62.3 | 89.1 | 94.7 |
| 1080 | 80.4 | 94.6 | 97.4 |
| 1260 | 90.1 | 98.5 | 100.3 |

FIG. 4 shows the delayed release profile of the pridopidine base formulation (PB-1) compared to the modified release formulations of pridopidine HCl (MR-1 and MR-2).

### Example 4

### Pridopidine Base Granulates

Tablet dosage forms of pridopidine base were prepared with granulates R1-R4. The granulates were prepared as described below.

Manufacture of Pridopidine Base Granulates:

High Shear Granulation: All granulation ingredients are added to the granulator bowl and pre-blend (chopper at medium/high speed; impeller at medium/low speed) for a sufficient time to ensure mixture uniformity and to break-up any agglomerates. Granulations liquid is added and blend (chopper at high speed; impeller at medium speed). The quantity of granulation fluid required is highly formulation dependent. The granules are dried using a fluid bed dryer and are milled by Quadro Comill.

Pridopidine granules (granulates) at 90 mg and higher dose pridopidine base are presented in Tables 12-14.

TABLE 12
Composition of Pridopidine Base Granules (Granulate) R1-R3

| | Batch No. | | | |
|---|---|---|---|---|
| | Use | R1 | R2 | R3 |
| Composition | — | mg/tab | mg/tab | mg/tab |
| Pridopidine base | Drug | 90 | 90 | 90 |
| | Substance | | | |
| Ethylcellulose (Ethocel ™ | Binder | 20.4 | 20.4 | 50.8 |
| 7 Premium) | | | | |
| CaHPO₄ | Insoluble | — | 178.0 | 101.6 |
| | filler | | | |
| Pregelatinized Starch (Starch | Filler, | — | — | 50.8 |
| 1500 ®) | disintegrant, | | | |
| | binder | | | |

TABLE 13
Composition of Pridopidine Base Granules (Granulate),
R4, based on High Dose IR Capsules formulation

| | Batch No. | |
|---|---|---|
| | Use | R4 |
| Composition | — | mg/Tab |
| Pridopidine base | Drug Substance | 112.5 |
| Microcrystalline Cellulose (Avicel ® | Diluent/disintegrant | 65.0 |
| PH 102) | | |
| Hydroxypropyl Cellulose (Klucel ™) | Binder | 10.0 |

TABLE 14
Composition of Pridopidine Base Granules (Granulate) R5

Batch No.

| | Use | R5 |
|---|---|---|
| Composition | — | mg/Tab |
| Pridopidine base | Drug Substance | 90 |
| Silicified Microcrystalline Cellulose | Filler | 63.2 |
| (Prosolv ® SMCC 90) | | |

Formulations

Pridopidine base modified release dosage forms formulations are described in Tables 15 and 16. These formulations provide a modified release similar to formulation PB-1 described above. Additionally, similar to Example 3, a special enteric coating (EC) polymer was added to the formulations of Tables 15 and 16 and a modified release similar the modified release of EC-PB-1 is obtained. The formulations of Tables 15 and 16 are also encapsulated in a special delayed release (DR) capsule similar to Example 3 and a modified release similar the modified release of DR-PB-1 is obtained.

The modified release dosage forms of pridopidine base formulations disclosed in Examples 3 and 4 are compared with the pharmacokinetics parameters of similar formulations containing pridopidine HCl. For example, AUC day1 0-38 h*ng/mL*h, Cmax*(ng/mL), AUC day1 0-50 h*ng/mL*h, AUCtau,ss (hr*ng/mL), and Cmax,ss (ng/mL) are determined for formulations of Examples 3 and 4 and with the same formulations except that pridopidine HCl is substituted for pridopidine base in a way that the amount of pridopidine is the same.

The modified release formulations of pridopidine base are found to be equal to or better than the modified release formulations of pridopidine HCl in terms of reducing the maximal blood concentration (Cₘₐₓ) while maintaining similar AUC levels.

TABLE 15

| | Composition (mg)/Prototype No. | |
|---|---|---|
| Formulation Ingredients | PB-2 | PB-3 |
| Pridopidine base | 90 | 90 |
| Silicified Microcrystalline Cellulose | 63.2 | 63.2 |
| (Prosolv ® SMCC 90) | | |
| Hydroxypropyl methylcellulose | ** | 150.0 |
| (Methocel ™ K100M Premium CR) | | |
| Hydrogenated Castor Oil (HCO) | 150.0 | ** |
| Lactose SD | 70.0 | 70.0 |
| Colloidal Silicon Dioxide (Aerosil ®) | 7.2 | 7.2 |
| Magnesium Stearate | 8.0 | 8.0 |
| Ethylcellulose (Surelease ®) | ** | 6.0-12.0 |

TABLE 16

| | No. | | | | | | | | |
|---|---|---|---|---|---|---|---|---|---|
| | Use | PB-4 | PB-5 | PB-6 | PB-7 | PB-8 | PB-9 | PB-10 | PB-11 |
| Composition | — | mg/Tab | mg/Tab | mg/Tab | mg/Tab | mg/Tab | mg/Tab | mg/Tab | mg/Tab |
| Granules (Granulate) | — | R1 | R4 | R3 | R3 | R1 | R1 | R2 | R4 |
| | | 110.4 | 187.5 | 293.2 | 293.2 | 110.4 | 110.4 | 288.4 | 151.5 |
| Calcium Phosphate Dibasic | Insoluble filler | * | * | * | * | 154.0 | * | * | * |
| Hydroxypropyl Methyl Cellulose | Hydrophilic | 122.0 | * | 90.0 | 90.0 | 120.0 | 120.0 | 90.0 | 150.0 |
| (HPMC) Methocel ™ K100 PR | carrier | | | | | | | | |
| CR | | | | | | | | | |
| (HPMC) | Hydrophilic | * | * | * | * | * | * | * | 25.0 |
| Methocel ™ K15M CR | carrier | | | | | | | | |
| Hydrogenated Castor Oil | Hydrophobic | 30.0 | 175.0 | * | 60.0 | * | * | * | * |
| | carrier | | | | | | | | |
| Aerosil ® | Flow agent | * | * | 5.0 | 5.0 | 2.0 | 2.0 | 5.0 | * |
| Mg. Stearate | Lubricant | 2.0 | 1.8 | 5.2 | 5.2 | 2.0 | 2.0 | 5.0 | 1.8 |
| LubriTose Blue¹ | Lubricant | * | 160.0 | * | * | * | * | * | * |
| LubriTose Yellow | Lubricant | * | * | * | * | * | * | * | 160.0 |
| Lactose Anhydrous | Soluble filler | * | * | 150.0 | 75.0 | * | 154.0 | 100 | * |

¹Lactose + (2%-10% Glyceryl MonoStearate): yellow contain 10% GMS and blue contain 2% GMS.

### Example 5

### Immediate Release (IR) Pridopidine Base Dosage Forms

In comparison to MR pridopidine base dosage forms, the IR dosage forms of pridopidine base dissolved in 0.1 N, HCl or acidic pH environment of the stomach. Examples of IR dosage forms of pridopidine base (22.5 mg and 45 mg) are presented in Table 17.

TABLE 17
Pridopidine Base IR Formulations

Batch No.

| | |
|---|---|
| I | II |

| | | |
|---|---|---|
| | | Composition |
| Formulation | Use | mg/capsule |

| | | | |
|---|---|---|---|
| Pridopidine base | Drug | 22.5 | 45 |
| | Substance | | |
| Silicified Microcrystalline | Filler | 43.2 | 86.4 |
| Cellulose (Prosolv ® SMCC 90) | | | |
| Magnesium Stearate | Lubricant | 1.4 | 2.8 |

### Example 6

### Synthesis of Pridopidine Base

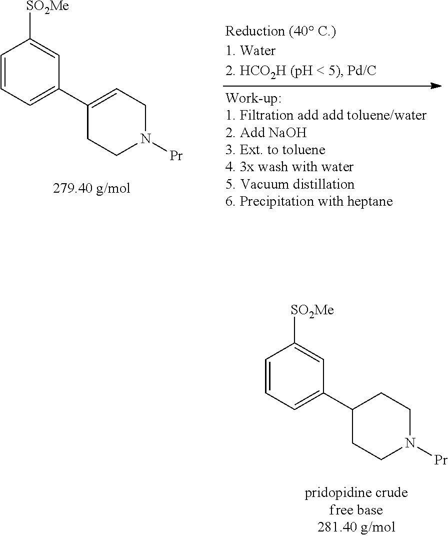

An example of method for making pridopidine free base is shown above. A process for making 4-(3-(methylsulfonyl)phenyl)-1-propyl-1,2,3,6-tetrahydropyridine free base is disclosed in U.S. Pat. No. 7,923,459. 4-(3-(methylsulfonyl)phenyl)-1-propyl-1,2,3,6-tetrahydropyridine free base is charged into reactor followed by 2.5 Vol of water and 0.2 Vol of formic acid in order to dissolve the raw material. The mixture is warmed and mixed at 30° C. (Tr=25-35° C.). The reactor is purged with nitrogen. 10% Pd/C wet (10% w/w) catalyst is charged follow by nitrogen wash (8-12% w/w Pd/C). 0.5 Vol of additional formic acid is added dropwise, keeping the temperature below 40° C. (slightly exothermic addition, Tr<50° C.). When the acid addition is finished, the black mixture is warmed with good stirring to 40° C. for reduction reaction (heterogenic system, Tr=25-45° C.). The mixture is mixed for not longer than 3.5 hr at 40° C. until the reaction has finished (reaction time could be between 2-50 hr). The conversion is analyzed by IPC. The reaction is run to completion when pridopidine ≧99.50%. When the reaction has completed, the mixture is filtered to remove the catalyst and washed with 2 Vol of water at Tr=25-35° C. The filtrate is transferred to another reactor with 5 Vol of toluene. The two phases are mixed together at 30° C. (Tr=25-35° C.). Aqueous sodium hydroxide solution (40%) is added dropwise until water phase pH is between 11-14 and then mixed for at least 30 min at 30° C. (Tr<50° C., −0.75 Vol, pH-13). The mixing is stopped for 20 min and the resulted clear yellow aqueous phase is removed. Three water washes are performed at 30° C. using 5 Vol each for purifying the product after which a pH of <10 is obtained. The reaction mixture is cooled down to 15° C. (Tr=10-20° C.) for vacuum distillation. The clear mixture is distilled under vacuum when the pressure is reduced to P<80 mbar, the Tj is carefully warm up to 60° C. until 2-2.5 Vol toluene remains in the reactor (Tc=0° C., Tr=15-45° C., Tj<70° C.). After the vacuum distillation has finished 4 Vol of n-heptane are added at 40° C. and heavy slurry is formed. The slurry is warmed to 50° C. and mixed for lhr for dissolution. The clear yellow solution is cooled down to 45° C. and mixed for 4 hr for crystallization. The crystallization mixture is cooled down to 0° C. for 4 hr and mixed for additional 4 hr. The solid is easily filtered and washed with 2 Vol n-heptane to remove the crust from the reactor walls. The wet cake is dried under vacuum (P<50 mbar) at 40° C. to constant weight. Dried pridopidine crude is obtained as yellow to white crystals solid and deliver 75%-90% yield. (Assay >98%, CP>99%).

### Example 7

Dosage forms of pridopidine are prepared according to Example 3 or 4, specifically PB-1, DR-PB-1, or EC-PB-1. Periodic oral administration of PB-1, DR-PB-1, or EC-PB-1 to a human patient afflicted with Huntington's Disease shows that the frequency of adverse events decreases compared to the frequency of adverse events in Example 1.

### Example 8

Dosage forms of pridopidine are prepared according to Example 3 or 4, specifically PB-1, DR-PB-1, or EC-PB-1, however the amount of pridopidine is 100 mg and each of the other components of PB-1, DR-PB-1, or EC-PB-1 are increased proportionally. Periodic oral administration of the dose forms to a human patient afflicted with Hungington's Disease shows that the Cₘₐₓ is no higher than previously tested safe doses.

### Example 9

Dosage forms of pridopidine are prepared according to Example 3 or 4, specifically PB-1, DR-PB-1, or EC-PB-1, however the amount of pridopidine is 125 mg and each of the other components of PB-1, DR-PB-1, or EC-PB-1 are increased proportionally. Periodic oral administration of the dose forms to a human patient afflicted with Huntington's Disease shows that the Cmax is no higher than previously tested safe doses.

### Example 10

Dosage forms of pridopidine are prepared according to Example 3 or 4, specifically PB-1, DR-PB-1, or EC-PB-1, however the amount of pridopidine is 135 mg and each of the other components of PB-1, DR-PB-1, or EC-PB-1 are increased proportionally. Periodic oral administration of the dose forms to a human patient afflicted with Hungington's Disease shows that the Cmax is no higher than previously tested safe doses.

### Example 11

Dosage forms of pridopidine are prepared according to Example 3 or 4, specifically PB-1, DR-PB-1, or EC-PB-1, however the amount of pridopidine is 150 mg and each of the other components of PB-1, DR-PB-1, or EC-PB-1 are increased proportionally. Periodic oral administration of the dose forms to a human patient afflicted with Huntington's Disease shows that the Cmax is no higher than previously tested safe doses.

### Example 12

Dosage forms of pridopidine are prepared according to Example 3 or 4, specifically PB-1, DR-PB-1, or EC-PB-1, however the amount of pridopidine is 180 mg and each of the other components of PB-1, DR-PB-1, or EC-PB-1 are increased proportionally. Periodic oral administration of the dose forms to a human patient afflicted with Huntington's Disease shows that the Cₘₐₓ is no higher than previously tested safe doses.

### Example 13

Dosage forms of pridopidine are prepared according to Example 3 or 4, specifically PB-1, DR-PB-1, or EC-PB-1, however the amount of pridopidine is 225 mg and each of the other components of PB-1, DR-PB-1, or EC-PB-1 are increased proportionally. Periodic oral administration of the dose forms to a human patient afflicted with Huntington's Disease shows that the Cmax is no higher than previously tested safe doses.

### Example 14

Dosage forms of pridopidine are prepared according to Example 3 or 4, specifically PB-1, DR-PB-1, or EC-PB-1, however the amount of pridopidine is more than 90 mg and at least one of the other components of PB-1, DR-PB-1, or EC-PB-1 is not increased proportionally. Therefore, the size of the dosage form is smaller than the size of the dosage form when all components are increased proportionally.

### Example 15

Dosage forms of pridopidine are prepared according to Example 3 or 4, specifically PB-1, DR-PB-1, or EC-PB-1, however the amount of pridopidine is more than 90 mg and at least one of the other components of PB-1, DR-PB-1, or EC-PB-1 is replaced with a different rate controlling excipient, mucoadhesive, binder, filler, plasticizer, glidant, lubricant, and/or diluent, so that the size of the dosage form is smaller than the size of the dosage form when the components of Example 3 are increased proportionally to the increase of the amount of pridopidine.

### Example 16

Dosage forms of pridopidine are prepared according to Example 3 or 4, specifically PB-1, DR-PB-1, or EC-PB-1, however the amount of pridopidine is 100 mg and at least one of the other components of PB-1, DR-PB-1, or EC-PB-1 is not increased proportionally. Therefore, the size of the dosage form is smaller than the size of the dosage form when all components are increased proportionally.

### Example 17

Dosage forms of pridopidine are prepared according to Example 3, specifically PB-1, DR-PB-1, or EC-PB-1, however the amount of pridopidine is 100 mg and at least one of the other components of PB-1, DR-PB-1, or EC-PB-1 is replaced with a different rate controlling excipient, mucoadhesive, binder, filler, plasticizer, glidant, lubricant, and/or diluent, so that the size of the dosage form is smaller than the size of the dosage form when the components of Example 3 are increased proportionally to the increase of the amount of pridopidine.

### Example 18

Dosage forms of pridopidine are prepared according to Example 3 or 4, specifically PB-1, DR-PB-1, or EC-PB-1, however the amount of pridopidine is 125 mg and at least one of the other components of PB-1, DR-PB-1, or EC-PB-1 is not increased proportionally. Therefore, the size of the dosage form is smaller than the size of the dosage form when all components are increased proportionally.

### Example 19

Dosage forms of pridopidine are prepared according to Example 3 or 4, specifically PB-1, DR-PB-1, or EC-PB-1, however the amount of pridopidine is 125 mg and at least one of the other components of PB-1, DR-PB-1, or EC-PB-1 is replaced with a different rate controlling excipient, mucoadhesive, binder, filler, plasticizer, glidant, lubricant, and/or diluent, so that the size of the dosage form is smaller than the size of the dosage form when the components of Example 3 are increased proportionally to the increase of the amount of pridopidine.

### Example 20

Dosage forms of pridopidine are prepared according to Example 3 or 4, specifically PB-1, DR-PB-1, or EC-PB-1, however the amount of pridopidine is 135 mg and at least one of the other components of PB-1, DR-PB-1, or EC-PB-1 is not increased proportionally. Therefore, the size of the dosage form is smaller than the size of the dosage form when all components are increased proportionally.

### Example 21

Dosage forms of pridopidine are prepared according to Example 3 or 4, specifically PB-1, DR-PB-1, or EC-PB-1, however the amount of pridopidine is 135 mg and at least one of the other components of PB-1, DR-PB-1, or EC-PB-1 is replaced with a different rate controlling excipient, mucoadhesive, binder, filler, plasticizer, glidant, lubricant, and/or diluent, so that the size of the dosage form is smaller than the size of the dosage form when the components of Example 3 are increased proportionally to the increase of the amount of pridopidine.

### Example 22

Dosage forms of pridopidine are prepared according to Example 3 or 4, specifically PB-1, DR-PB-1, or EC-PB-1, however the amount of pridopidine is 150 mg and at least one of the other components of PB-1, DR-PB-1, or EC-PB-1 is not increased proportionally. Therefore, the size of the dosage form is smaller than the size of the dosage form when all components are increased proportionally.

### Example 23

Dosage forms of pridopidine are prepared according to Example 3 or 4, specifically PB-1, DR-PB-1, or EC-PB-1, however the amount of pridopidine is 150 mg and at least one of the other components of PB-1, DR-PB-1, or EC-PB-1 is replaced with a different rate controlling excipient, mucoadhesive, binder, filler, plasticizer, glidant, lubricant, and/or diluent, so that the size of the dosage form is smaller than the size of the dosage form when the components of Example 3 are increased proportionally to the increase of the amount of pridopidine.

### Example 24

Dosage forms of pridopidine are prepared according to Example 3 or 4, specifically PB-1, DR-PB-1, or EC-PB-1, however the amount of pridopidine is 180 mg and at least one of the other components of PB-1, DR-PB-1, or EC-PB-1 is not increased proportionally. Therefore, the size of the dosage form is smaller than the size of the dosage form when all components are increased proportionally.

### Example 25

Dosage forms of pridopidine are prepared according to Example 3 or 4, specifically PB-1, DR-PB-1, or EC-PB-1, however the amount of pridopidine is 180 mg and at least one of the other components of PB-1, DR-PB-1, or EC-PB-1 is replaced with a different rate controlling excipient, mucoadhesive, binder, filler, plasticizer, glidant, lubricant, and/or diluent, so that the size of the dosage form is smaller than the size of the dosage form when the components of Example 3 are increased proportionally to the increase of the amount of pridopidine.

### Example 26

Dosage forms of pridopidine are prepared according to Example 3 or 4, specifically PB-1, DR-PB-1, or EC-PB-1, however the amount of pridopidine is 225 mg and at least one of the other components of PB-1, DR-PB-1, or EC-PB-1 is not increased proportionally. Therefore, the size of the dosage form is smaller than the size of the dosage form when all components are increased proportionally.

### Example 27

Dosage forms of pridopidine are prepared according to Example 3 or 4, specifically PB-1, DR-PB-1, or EC-PB-1, however the amount of pridopidine is 225 mg and at least one of the other components of PB-1, DR-PB-1, or EC-PB-1 is replaced with a different rate controlling excipient, mucoadhesive, binder, filler, plasticizer, glidant, lubricant, and/or diluent, so that the size of the dosage form is smaller than the size of the dosage form when the components of Example 3 are increased proportionally to the increase of the amount of pridopidine.

### REFERENCES

Clinicaltrials.gov Clinical Trial Identifier NCT02006472, “A Phase 2, to Evaluating the Safety and Efficacy of Pridopidine Versus Placebo for Symptomatic Treatment in Patients With Huntington's Disease.”
CSID:25948790, http://www.chemspider.com/Chemical-Structure.25948790.html (accessed 23:27, Jul. 15, 2016).
CSID:7971505, http://www.chemspider.com/Chemical-Structure.7971505.html (accessed 23:33, Jul. 15, 2016).
de Yebenes J G, Landwehrmeyer B, Squitieri F, Reilmann R, Rosser A, Barker R A, Saft C, Magnet M K, Sword A, Rembratt A, Tedroff J; Mermai H D study investigators, “Pridopidine for the treatment of motor function in patients with Huntington's disease (MermaiHD): a phase 3, randomised, double-blind, placebo-controlled trial,” Lancet Neurol. 2011 December; 10(12):1049-57. doi: 10.1016/S1474-4422(11)70233-2. Epub 2011 Nov. 7.
Huntington Study Group HART Investigators, “A randomized, double-blind, placebo-controlled trial of pridopidine in Huntington's disease,” Mov Disord. 2013 September; 28(10):1407-15. doi: 10.1002/mds.25362. Epub 2013 Feb. 28.
Helldén A, Panagiotidis G, Johansson P, Waters N, Waters S, Tedroff J, Bertilsson L. “The dopaminergic stabilizer pridopidine is to a major extent N-depropylated by CYP2D6 in humans” Eur J Clin Pharmacol. 2012 September; 68(9):1281-6. Epub 2012 Mar. 8.
Lindskov Krog P, Osterberg O, Gundorf Drewes P, Rembratt A, Schultz A, Timmer W. “Pharmacokinetic and tolerability profile of pridopidine in healthy-volunteer poor and extensive CYP2D6 metabolizers, following single and multiple dosing” Eur J Drug Metab Pharmacokinet. 2013 March; 38(1):43-51. Epub 2012 Sep. 5.
Osterberg, et al. “A single center, randomized, placebo-controlled, double-blind study to evaluate the safety, tolerability, and pharmacokinetics of multiple-ascending doses of pridopidine in healthy volunteers” Poster presented at Sixth Annual Huntington Disease Clinical Research Symposium, November 2012, Seattle, Wash., USA. Neurotherapeutics
Medical Dictionary for the Health Professions and Nursing, Farlex 2012
Evonik Industries, “Eudragit® L30 D-55” http://eudragit.evonik.com/product/eudragit/en/products-services/eudragit-products/enteric-formulations/1-30-d-55/pages/default.aspx, accessed Jun. 17, 2015, cited above as Evonik I.
Evonik Industries, “Eudragit® L100 D-55” http://eudragit.evonik.com/product/eudragit/en/products-services/eudragit-products/enteric-formulations/1-100-55/pages/default.aspx, accessed Jun. 17, 2015, cited above as Evonik II.

1. A modified release solid oral dosage form comprising a therapeutically effective amount of pridopidine base, and at least one pharmaceutically acceptable rate controlling excipient.

2. The modified release solid oral dosage form of claim 1, wherein the solid oral dosage form provides
(a) an in vivo plasma pridopidine concentration profile having a Mean Cₘₐₓ of about 1,400 ng/ml or less,
(b) an in vivo plasma pridopidine concentration profile having a Mean Cₘₐₓ of a) about 1,157 ng/ml or less; b) about 906 ng/ml or less; or c) about 499 ng/ml or less,
(c) an in vivo plasma pridopidine concentration profile having a Mean Cmax of: a) about 718 ng/ml or less measured after single dose administration; b) about 486 ng/ml or less measured after single dose administration; or c) about 327 ng/ml or less measured after single dose administration,
(d) an in vivo plasma pridopidine concentration profile having a Cmax a) from about 382 ng/ml to about 1,568 ng/ml; b) between 871 ng/ml and 1,568 ng/ml; c) between 382 ng/ml and 1,287 ng/ml; or d) between 639 ng/ml and 1,287 ng/ml, or
(e) an in vivo plasma pridopidine concentration profile having a Cₘₐₓ a) from about 244 ng/ml to about 1,002 ng/ml; b) between 244 ng/ml and 813 ng/ml; c) between 493 ng/ml and 1,002 ng/ml; or d) between 324 ng/ml and 813 ng/ml.

3-6. (canceled)

7. The modified release solid oral dosage forms of claim 2, wherein
(a) the AUCₜₐᵤ is about 5,253 ng h/ml or more,
(b) the AUC_{0-inf} is about 2,249 ng h/ml or more,
(c) the Mean AUCₜₐᵤ is a) about 7,178 ng h/ml or more; b) about 14,185 ng h/ml or more; or c) about 18,065 ng h/ml or more,
(d) the Mean AUC_{0-inf} is about a) 5,043 ng h/ml or more; b) about 7,897 ng h/ml or more; or c) about 13,594 ng h/ml or more,
(e) the in vivo plasma profile is measured at steady state,
(f) the in vivo plasma profile is measured after single dose administration, or
(g) AUC_{inf} is estimated from AUC₀₋₂₄.

8-13. (canceled)

14. A modified release solid oral dosage form comprising a therapeutically effective amount of pridopidine base, and at least one pharmaceutically acceptable rate controlling excipient, and wherein the solid oral dosage form provides an in vivo plasma pridopidine concentration profile having a Mean Cmax which is lower than a Mean Cmax resulting from the b.i.d. administration of an immediate release solid oral dosage form which contains:
a) half the amount of the pridopidine; or
b) between 10% and 49% of the amount of the pridopidine, optionally,
(i) wherein a) the amount of pridopidine base is more than 45 mg of pridopidine; b) the amount of pridopidine base in the modified release dosage form is at least about 90 mg of pridopidine and the immediate release dosage form contains about 45 mg of pridopidine; c) the amount of pridopidine base in the modified release dosage form is at least about 100 mg of pridopidine and the immediate release solid oral dosage form contains about 45 mg of pridopidine; d) the amount of pridopidine base in the modified release dosage form is at least about 125 mg of pridopidine and the immediate release solid oral dosage form contains about 45 mg of pridopidine; e) the amount of pridopidine base in the modified release dosage form is at least about 135 mg of pridopidine and the immediate release solid oral dosage form contains about 45 mg of pridopidine; f) the amount of pridopidine base in the modified release dosage form is at least about 135 mg of pridopidine and the immediate release solid oral dosage form contains about 67.5 mg of pridopidine; g) the amount of pridopidine base in the modified release dosage form is at least about 150 mg of pridopidine and the immediate release solid oral dosage form contains about 45 mg of pridopidine; h) the amount of pridopidine base in the modified release dosage form is at least about 150 mg of pridopidine and the immediate release solid oral dosage form contains about 67.5 mg of pridopidine; i) the amount of pridopidine base in the modified release dosage form is at least about 180 mg of pridopidine and the immediate release solid oral dosage form contains about 45 mg of pridopidine; j) the amount of pridopidine base in the modified release dosage form is at least about 180 mg of pridopidine and the immediate release solid oral dosage form contains about 67.5 mg of pridopidine; k) the amount of pridopidine base in the modified release dosage form is at least about 180 mg of pridopidine and the immediate release solid oral dosage form contains about 90 mg of pridopidine; 1) the amount of pridopidine base in the modified release dosage form is at least about 200 mg of pridopidine and the immediate release solid oral dosage form contains about 45 mg of pridopidine; m) the amount of pridopidine base in the modified release dosage form is at least about 200 mg of pridopidine and the immediate release solid oral dosage form contains about 67.5 mg of pridopidine; n) the amount of pridopidine base in the modified release dosage form is at least about 200 mg of pridopidine and the immediate release solid oral dosage form contains about 90 mg of pridopidine; o) the amount of pridopidine base in the modified release dosage form is at least about 225 mg of pridopidine and the immediate release solid oral dosage form contains about 45 mg of pridopidine; p) the amount of pridopidine base in the modified release dosage form is at least about 225 mg of pridopidine and the immediate release solid oral dosage form contains about 67.5 mg of pridopidine; q) the amount of pridopidine base in the modified release dosage form is at least about 225 mg of pridopidine and the immediate release solid oral dosage form contains about 90 mg of pridopidine; r) the amount of pridopidine base in the modified release dosage form is at least about 225 mg of pridopidine and the immediate release solid oral dosage form contains about 112.5 mg of pridopidine; s) the amount of pridopidine base in the modified release dosage form is at least about 250 mg of pridopidine and the immediate release solid oral dosage form contains about 45 mg of pridopidine; t) the amount of pridopidine base in the modified release dosage form is at least about 250 mg of pridopidine and the immediate release solid oral dosage form contains about 67.5 mg of pridopidine; u) the amount of pridopidine base in the modified release dosage form is at least about 250 mg of pridopidine and the immediate release solid oral dosage form contains about 90 mg of pridopidine; v) the amount of pridopidine base in the modified release dosage form is at least about 250 mg of pridopidine and the immediate release solid oral dosage form contains about 112.5 mg of pridopidine; w) the amount of pridopidine base in the modified release dosage form is at least about 315 mg of pridopidine and the immediate release solid oral dosage form contains about 45 mg of pridopidine; x) the amount of pridopidine base in the modified release dosage form pridopidine is at least about 315 mg of pridopidine and the immediate release solid oral dosage form contains about 67.5 mg of pridopidine; y) the amount of pridopidine base in the modified release dosage form is at least about 315 mg of pridopidine and the immediate release solid oral dosage form contains about 90 mg of pridopidine; z) the amount of pridopidine base in the modified release dosage form is at least about 315 mg of pridopidine and the immediate release solid oral dosage form contains about 112.5 mg of pridopidine; or aa) the amount of pridopidine base in the modified release dosage form is at least about 315 mg of pridopidine and the immediate release solid oral dosage form contains about 157.5 mg of pridopidine,
(ii) wherein a) the solid oral dosage form provides an in vivo plasma pridopidine concentration profile having a Mean AUCtau which is at least about 50% of the Mean AUCₜₐᵤ provided by the b.i.d. administration of an immediate release solid oral dosage form which contains half the amount of the pridopidine; b) the solid oral dosage form provides an in vivo plasma pridopidine concentration profile having a Mean AUCₜₐᵤ which is at least about 60% of the Mean AUCₜₐᵤ provided by the b.i.d. administration of an immediate release solid oral dosage form which contains half the amount of the pridopidine; c) the solid oral dosage form provides an in vivo plasma pridopidine concentration profile having a Mean AUCₜₐᵤ which is at least about 70% of the Mean AUCₜₐᵤ provided by the b.i.d. administration of an immediate release solid oral dosage form which contains half the amount of the pridopidine; d) the solid oral dosage form provides an in vivo plasma pridopidine concentration profile having a Mean AUCtau which is at least about 80% of the Mean AUCₜₐᵤ provided by the b.i.d. administration of an immediate release solid oral dosage form which contains half the amount of the pridopidine; e) the solid oral dosage form provides an in vivo plasma pridopidine concentration profile having a Mean AUCtau which is at least about 90% of the Mean AUCtau provided by the b.i.d. administration of an immediate release solid oral dosage form which contains half the amount of the pridopidine; or f) the solid oral dosage form provides an in vivo plasma pridopidine concentration profile having a Mean AUCₜₐᵤ which is at least about 95% of the Mean AUCtau provided by the b.i.d. administration of an immediate release solid oral dosage form which contains half the amount of the pridopidine, or
(iii) wherein the b.i.d. administration of an immediate release solid oral dosage form has a time interval between doses of 5-10 hours, 6-8 hours, 6.5 hours, or 7 hours.

15-17. (canceled)

18. The modified release solid oral dosage form of claim 1, wherein the solid oral dosage form provides an in vivo plasma pridopidine concentration profile having a Mean Cₘₐₓ which is reduced by a percentage compared to the Mean Cmax resulting from the b.i.d. administration of an immediate release dosage form which contains half the amount of the pridopidine wherein the percentage is at least 5%, preferably a) at least 10%; b) at least 20%; c) at least 30%; d) at least 40%; e) at least 50%; f) at least 60%; g) at least 70%; h) between 10% and 60%; i) between 20% and 50%; j) about 25%; k) about 35%; or l) about 50%.

19. (canceled)

20. The modified release solid oral dosage form of claim 14, wherein
(a) the mean time required to reach the maximal plasma, serum or blood concentration of the drug, following administration of the drug is more than 2 hours or more than 4 hours,
(b) the in vivo plasma profile is measured at steady state,
(c) the in vivo plasma profile is measured after single dose administration, preferably wherein a) the solid oral dosage form provides an in vivo plasma pridopidine concentration profile having a mean AUC_{0-inf} which is at least about 50% of the mean AUC_{0-inf} provided by the b.i.d. administration of an immediate release solid oral dosage form which contains half the amount of the pridopidine; b) the solid oral dosage form provides an in vivo plasma pridopidine concentration profile having a mean AUC_{0-inf} which is at least about 55% of the mean AUC_{0-inf} provided by the b.i.d. administration of an immediate release solid oral dosage form which contains half the amount of the pridopidine; or c) the solid oral dosage form provides an in vivo plasma pridopidine concentration profile having a mean AUC_{0-inf} which is at least about 75% of the mean AUC_{0-inf} provided by the b.i.d. administration of an immediate release solid oral dosage form which contains half the amount of the pridopidine.

21-23. (canceled)

24. The modified release solid oral dosage form of claim 1, wherein
(a) the modified release solid oral dosage form releases 1-20%, 1-15%, 1-10%, 5%-15%, or 5%-10% of pridopidine after 1 hour when the oral dosage form is placed in an apparatus comprising phosphate buffer having a pH of 6.8,
(b) the modified release solid oral dosage form releases 1-50%, 5-45%, 10-40%, 10-35%, 10-25%, 10-20% or 15-20% of pridopidine after 3 hours when the oral dosage form is placed in an apparatus comprising phosphate buffer having a pH of 6.8,
(c) the modified release solid oral dosage form releases 1-70%, 10-60%, 20-50%, 20-45%, 20-40%, 20-35%, or about 25% of pridopidine after 6 hours when the oral dosage form is placed in an apparatus comprising phosphate buffer having a pH of 6.8,
(d) the modified release solid oral dosage form releases 1-85%, 15-60%, 30-75%, 40-55%, 40-50%, 30-50% or about 48.3% of pridopidine after 9 hours when the oral dosage form is placed in an apparatus comprising phosphate buffer having a pH of 6.8,
(e) the modified release solid oral dosage form releases 1-95%, 15-90%, 30-80%, 50-70%, 55-65% or about 61% of pridopidine after 12 hours when the solid oral dosage form is placed in an apparatus comprising phosphate buffer having a pH of 6.8,
(f) the modified release solid oral dosage form releases 0-10%, 0-25%, 0-30%, or 0.5-10%, 7%, or 2.5% of pridopidine after 1 hour when the oral dosage form is placed in an apparatus comprising an acidic medium for one hours,
(g) the modified release solid oral dosage form releases 5%-45% or 5%-30% or 0%-10% or 20%-50% or about 20.5%, or about 7.0% of pridopidine after 2 hours when the oral dosage form is placed in an apparatus comprising an acidic medium for two hours,
(h) the modified release solid oral dosage form releases 1-75%, 3-75%, or 40-60% of pridopidine after 6 hours when the oral dosage form is placed in an apparatus comprising an acidic medium for two hours and then in a phosphate buffer having a pH of 6.8 for 4 hours,
(i) the modified release solid oral dosage form releases 1-90%, 15-75%, or 50-75% of pridopidine after 8 hours when the oral dosage form is placed in an apparatus comprising an acidic medium for two hours and then in a phosphate buffer having a pH of 6.8 for 6 hours,
(j) wherein the modified release solid oral dosage form releases 1-90%, or 60-85% of pridopidine after 10 hours when the oral dosage form is placed in an apparatus comprising an acidic medium for two hours and then in a phosphate buffer having a pH of 6.8 for 8 hours,
(k) the modified release solid oral dosage form releases 1-95%, or 60-95% of pridopidine after 12 hours when the oral dosage form is placed in an apparatus comprising an acidic medium for two hours and then in a phosphate buffer having a pH of 6.8 for 10 hours,
(l) the modified release solid oral dosage form releases less pridopidine after 6 hour when placed in an apparatus comprising phosphate buffer having a pH of 6.8, than a formulation consisting of pridopidine HCl and the same rate controlling excipients when placed under the same conditions, wherein the amount of pridopidine and the amount of rate controlling excipients are the same in the solid oral dosage form and the formulation,
(m) the modified release solid oral dosage form releases less pridopidine after 6 hour when placed in an apparatus comprising phosphate buffer having a pH of 6.8, than a formulation consisting of pridopidine HCl and the same rate controlling excipients when placed under the same conditions, wherein the amount of pridopidine and the amount of rate controlling excipients are the same in the solid oral dosage form and the formulation, or
(n) the solid oral dosage form releases less pridopidine after 6 hours, after 9 hours, or after 12 hours when placed in an apparatus comprising an acidic medium for two hours and then in a phosphate buffer having a pH of 6.8, than a formulation consisting of pridopidine HCl and the same rate controlling excipients when placed under the same conditions, wherein the amount of pridopidine and the amount of rate controlling excipients are the same in the solid oral dosage form and the formulation, and
preferably wherein the apparatus is a basket and/or paddle apparatus and is maintained at a temperature of 37° C. rotating at 50-100 revolutions per minute, more preferably the acidic medium is not more than 1000 ml of HCl 0.1N or gastric fluid, more preferably the acidic medium is 500 mL of HCl 0.1N, most preferably the apparatus is a basket apparatus maintained at a temperature of 37° C. rotating at 100 revolutions per minute.

25-40. (canceled)

41. The modified release solid oral dosage form of claim 1, wherein
(a) the modified release solid dosage form comprises from, from about 45 mg to about 300 mg, or from about 90 mg to about 250 mg pridopidine,
(b) the modified release solid dosage form comprises at least about 90 mg, at least about 100 mg, at least about 125 mg, at least about 135 mg, at least about 150 mg, at least about 180 mg, at least about 200 mg, at least about 225 mg, at least about 250 mg, or at least about 315 mg, pridopidine,
(c) the dosage form comprises about 90 mg, about 100 mg, about 125 mg, about 135 mg, about 150 mg, about 180 mg, about 200 mg, about 225 mg, about 250 mg, or about 315 mg, pridopidine,
(d) the pridopidine comprises from about 15% to about 60%, about 25% to about 50%, about 20% to about 25%, about 20%, or about 25%, by weight of the modified release solid dosage form, and/or
(e) the modified release solid dosage form is in the form of a capsule, tablet, a mini tablet, or a pellet.

42-46. (canceled)

47. The modified release solid oral dosage form according to claim 1, wherein the rate controlling excipient is a polymeric material, preferably selected from a group consisting of a hydrophilic and a hydrophobic polymeric material, more preferably selected from a group consisting of: hydrogenated castor oil, polyethylene oxide, ethyl cellulose hydroxypropyl methylcellulose (HPMC), hydroxypropyl cellulose (HPC), polyvinyl alcohol (PVA), vinyl alcohol polymer, polycrylates, polymethacrylates, ethyl acrylate-methyl methacrylate copolymers, glyceryl monostearate, and mixtures thereof, more preferably the rate controlling excipient is a combination of two or more polymeric materials, most preferably the rate controlling excipient is hydroxypropyl methylcellulose and/or hydrogenated castor oil.

48-51. (canceled)

52. The modified release solid oral dosage form according to claim 1, wherein
(a) the total amount of the rate controlling excipients is from about 8% to about 70% of the total weight of the modified release solid dosage form, from about 10% to about 50% of the total weight of the modified release solid dosage form, or from about 20% to about 50% of the total weight of the modified release solid dosage form, from about 30% to about 50% or from about 30% to about 40% of the total weight of the modified release solid dosage form,
(b) the polymeric material is between 10% and 50%, between 20% and 50%, between 30% and 50%, between 30% and 40%, between 35% and 40%, at least 10%, at least 20%, at least 25%, at least 30%, at least 35%, at least 40%, about 33%, about 36%, about 37%, about 38%, or about 40%, by weight of the modified release solid oral dose form,
(c) the polymeric material is hydroxypropyl methylcellulose or hydrogenated castor oil, and wherein the hydroxypropyl methylcellulose or hydrogenated castor oil is 33-38% by weight of the solid oral dose form, or
(d) wherein the weight ratio of the pridopidine base to the rate controlling excipient is from about 0.2:1 to about 1:1, about 0.3:1 to about 0.8:1, preferably about 0.5:1 to about 0.7:1.

53-55. (canceled)

56. The modified release solid oral dosage form of claim 1 further comprising a mucoadhesive, optionally, wherein the weight ratio of the pridopidine base to the rate controlling excipient is from about 0.2:1 to about 1:1, about 0.3:1 to about 0.8:1, preferably about 0.5:1 to about 0.7:1.

57-58. (canceled)

59. A pharmaceutical formulation comprising the modified release solid oral dosage form of claim 1, and one or more pharmaceutically acceptable carriers or excipients, preferably wherein the pharmaceutically acceptable carriers or excipients are selected from a group consisting of: binder, filler, plasticizer, glidant and lubricant, diluent, and mixtures thereof.

60. (canceled)

61. The pharmaceutical formulation according to claim 59, wherein
(a) the binder is selected from a group consisting of: starch, pregelatinized starch, polyethylene oxide, cellulose polymers, hydroxypropylmethyl cellulose, hydroxypropylcellulose, methylcellulose, hydroxyethyl cellulose, polyvinylpyrrolidone, polyvinyl alcohol and mixtures thereof, or
(b) the filler is selected from a group consisting of: microcrystalline cellulose, sugar spheres, lactose, sorbitol, dextrose, sucrose, mannitol, dibasic or tribasic calcium phosphate, calcium sulfate, starch, retalac and mixtures thereof, preferably wherein the filler or diluent is microcrystalline cellulose, lactose, silicified microcrystalline cellulose, a mixture of microcrystalline cellulose and lactose, more preferably wherein the filler is present in an amount of between 5% and about 64% by weight of the modified release solid oral dose form, between 10% and about 50% by weight of the modified release solid oral dose form, between 15% and about 45% by weight of the modified release solid oral dose form, between 20% and 40% by weight of the modified release solid oral dose form, between 29 and 34% by weight of the modified release solid oral dose form, about 34% by weight of the modified release solid oral dose form, about 16% by weight of the modified release solid oral dose form, about 17% by weight of the modified release solid oral dose form or about 18% by weight of the modified release solid oral dose form, most preferably the filler is a mixture of silicified microcrystalline cellulose and lactose and wherein silicified microcrystalline cellulose is between about 14% and about 16% by weight of the modified release solid oral dose form and lactose is between about 15% and about 18% by weight of the modified release solid oral dose form, optionally wherein the lactose is Lactose anhydrous or Lactose SD, DC.

62-69. (canceled)

70. The pharmaceutical formulation according to claim 59, wherein the plasticizer is selected from a group consisting of: polyethylene glycol, triethyl citrate, tributyl citrate, glycerin, dibutyl sebacate, triacetin, diethylphthalat and mixtures thereof, preferably triethyl citrate.

71. (canceled)

72. The pharmaceutical formulation according to claim 59, wherein the glidant is selected from a group consisting of: starch, pregelatinized starch, silicone dioxide, colloidal silicone dioxide, talc and mixtures thereof, preferably the glidant is colloidal silicone dioxide, more preferably wherein the glidant is present in an amount of between 0.2% and about 4% by weight of the modified release solid oral dose form, between 0.4% and about 3% by weight of the modified release solid oral dose form, or between 0.43% and about 2% by weight of the modified release solid oral dose form, more preferably wherein the glidant is present in an amount of between 1.7% and about 4% by weight of the modified release solid oral dose form, between 1.7% and about 3% by weight of the modified release solid oral dose form, between 1.7% and about 2.0% by weight of the modified release solid oral dose form, between 1.7% and 1.8% by weight of the modified release solid oral dose form, about 1.5% by weight of the modified release solid oral dose form, about 1.7% by weight of the modified release solid oral dose form or about 1.8% by weight of the modified release solid oral dose form.

73-75. (canceled)

76. The pharmaceutical formulation according to claim 59, wherein the lubricant is selected from a group consisting of: sodium stearyl fumarate, stearic acid, magnesium stearate, calcium stearate, zinc stearate, talc, glyceryl behenate, glyceryl monostearate, and mixtures thereof, preferably the lubricant is magnesium stearate, more preferably the lubricant is between 0.3% and about 4% by weight of the modified release solid oral dose form, between 0.5% and about 3% by weight of the modified release solid oral dose form, or between 1.1% and about 2% by weight of the modified release solid oral dose form, more preferably the lubricant is between 1.7% and about 4% by weight of the modified release solid oral dose form, between 1.7% and about 3% by weight of the modified release solid oral dose form, between 1.7% and about 2.3% by weight of the modified release solid oral dose form, between 1.8% and about 2.2% by weight of the modified release solid oral dose form, about 1.8% by weight of the modified release solid oral dose form, about 1.9% by weight of the modified release solid oral dose form or about 2.0% by weight of the modified release solid oral dose form.

77-79. (canceled)

80. The modified release solid oral dose form of claim 1, wherein the modified release solid oral dose form is a tablet and the tablet further comprises an acid resistant envelope.

81. An enteric coated tablet comprising a core comprising the pharmaceutical formulation of any one of claims 59-79, and an overcoat layer, wherein the overcoat layer completely surrounds the core, optionally
(a) wherein the overcoat layer comprising a pH sensitive polymer barrier,
(b) wherein the overcoat layer comprises a coating suspension,
(c) wherein the overcoat layer comprises an anionic acrylic polymer,
(d) wherein the overcoat layer comprises an anionic polymer with methacrylic acid as a functional group,
(e) wherein the overcoat layer comprises an anionic polymer with methacrylic acid as a functional group,
(f) wherein the overcoat layer comprises a methacrylic Acid-Methyl Methacrylate Copolymer [1:1] or a solid poly(methacylic acid-co-ethyl acrylate) 1:1,
(g) wherein the overcoat layer dissolves slowly in the stomach, but dissolves quickly in the small intestine,
(h) wherein the overcoat layer comprises a lubricant, preferably the lubricant is talc, stearic acid, magnesium stearate, more preferably the lubricant is between 0.5% and about 4% by weight of the modified release solid oral dose form, between 1.5% and about 2% by weight of the modified release solid oral dose form or about 1.7% by weight of the modified release solid oral dose form,
(i) wherein the overcoat layer comprises a plasticizer, preferably the plasticizer is triethyl citrate, more preferably the plasticizer is present in an amount of between 0.2% and about 2.0% by weight of the modified release solid oral dose form, between 0.5% and about 1.0% by weight of the modified release solid oral dose form or about 0.7% by weight of the modified release solid oral dose form,
(j) wherein the enteric coated tablet or the delayed release capsule imparts protection to the core so that said core is afforded protection in a low pH environment of 3 or less while capable of releasing medicament at a pH of 6.0 or higher,
(k) wherein said tablet can withstand agitation in a basket at 100 rpm in artificial gastric juice having a pH of 1.2 at a temperature of 37° C. releasing less than 10% pridopidine in two hours.

82-93. (canceled)

94. A delayed release capsule comprising a core comprising the pharmaceutical formulation of claim 59, wherein the delayed release capsule completely surrounds the core, preferably wherein said capsule can withstand agitation in a basket at 100 rpm in artificial gastric juice having a pH of 1.2 at a temperature of 37° C. releasing less than 10% pridopidine in two hours.

95-98. (canceled)

99. A method of treating a subject afflicted with a condition selected from Huntington's Disease, Parkinson's disease, iatrogenic and non-iatrogenic Parkinsonism, dyskinesias, dystonias, Tourette's disease, iatrogenic and non-iatrogenic psychoses and hallucinoses, schizophrenia disorder or schizophreniform disorder, mood and anxiety disorders, manic depressive illness, depression, obsessive-compulsive disease, a sleep disorder, autism spectrum disorder, ADHD, age-related cognitive impairment, abuse of alcohol and substances used as narcotics, Alzheimer's disease and Retts syndrome, wherein the method comprises administering the modified release solid oral dosage form according to claim 1 or a method of treating an individual afflicted with a neurodegenerative disease or a disease related to dopamine, comprising once daily administration of the modified release solid oral dosage form.

100-102. (canceled)
